(19) United States
(12) Patent Application Publication
(10) Pub. No.: US 2012/0021039 A1
(43) Pub. Date: Jan. 26, 2012

(54) EXPRESSION OF NEUROPEPTIDES IN MAMMALIAN CELLS

(75) Inventors: Philip Kusk, Lynge (DK); Lars Ulrik Wahlberg, Tiverton, RI (US); Teit E. Johansen, Horsholm (DK)

(73) Assignee: NsGene A/S, Ballerup (DK)

(21) Appl. No.: 13/145,664

(22) PCT Filed: Jan. 21, 2010
(86) PCT No.: PCT/DK2010/050014

§ 371 Date: Oct. 11, 2011

Related U.S. Application Data
(60) Provisional application No. 61/146,754, filed on Jan. 23, 2009.

(30) Foreign Application Priority Data
Jan. 23, 2009 (PA) 2009 00103
Sep. 22, 2009 (PA) 2009 70122

(51) Int. Cl. A61F 2/00; C12N 5/10; A61K 48/00; A61K 38/17; A61P 25/04; A61P 25/08; A61P 25/00; A61P 25/24; A61P 25/22; A61P 25/18; C12N 15/85; A61P 25/28

(52) U.S. Cl. 424/424; 435/320.1; 435/325; 435/366; 435/358; 435/369; 435/365; 435/353; 435/352; 424/93.21

(57) ABSTRACT

The present application relates to expression constructs capable of securing correct processing of neuropeptides upon expression in mammalian cells, and to mammalian cells secreting correctly processed peptides. One exemplary peptide is galanin. The application also relates to devices containing neuropeptide secreting cells, which devices may be used for the treatment of epilepsy and other disorders of the nervous system. All references cited herein are incorporated by reference.

The present application relates to expression constructs capable of securing correct processing of neuropeptides upon expression in mammalian cells, and to mammalian cells secreting correctly processed peptides. One exemplary peptide is galanin. The application also relates to devices containing neuropeptide-secreting cells; such devices may be used for the treatment of epilepsy and other disorders of the nervous system. All references cited herein are incorporated by reference.

### BACKGROUND

Long-term delivery of neuropeptides to the central nervous system behind the blood-brain barrier may be accomplished in different ways: continuous infusion using implanted pumps or cannulae, in vivo gene therapy, or transplantation of naked cells that have been genetically modified to secrete the neuropeptide.

Delivery using implanted pumps or cannulae requires repeated infusions into the brain, either through injections via a cannula, or from pumps, which must be refilled every time the reservoir is depleted. Every occasion in which the pump reservoir must be replaced or the injection syringe reinserted through the cannulae represents another opportunity that contaminants might be introduced into the brain, which is especially susceptible to infection. Even with the careful use of sterile procedures, there is risk of infection. In addition to the risk of infection, there seems to be some risk associated with the infusion procedure. Infusions into the ventricles may produce hydrocephalus and continuous infusions of solutions into the parenchyma are associated with cell necrosis in the brain.

In vivo gene therapy is a promising technique for delivery of peptides to the central nervous system. It carries the advantage of in-situ synthesis of the active neuropeptide. However, gene therapy requires the use of virus vectors which use is inherently associated with risks of insertional mutagenesis and tumorigenesis as well as the inability to stop the neuropeptide secretion should untoward effects occur.

Neuropeptides are expressed by specialised neurons and neuroendocrine cells. These cells possess a specialised protein processing apparatus. Recombinant expression of neuropeptides in mammalian production cell lines therefore often leads to incorrect or incomplete processing of the protein precursors thus leading to non-bioactive neuropeptides. Thus, it is an object of the present invention to provide expression constructs that can lead to correctly processed neuropeptides in non-neurons and non-endocrine cells, such as production cells and therapeutic cells and cells targeted with a gene therapy vector.

### SUMMARY OF THE INVENTION

In a first aspect the invention relates to an expression construct for expression in a mammalian cell, the expression construct coding for a heterologous polypeptide comprising at least 51 amino acids, said heterologous polypeptide comprising from the N-terminal to the C-terminal a mammalian signal peptide, a pro-peptide, and a neuropeptide, wherein said neuropeptide is cleavable from the pro-peptide by furin at a furin-site, and wherein said furin-site is optimal for cleavage.

The construct may additionally encode a C-terminal peptide.

These expression constructs ensure correct processing and secretion of the encoded neuropeptide in a mammalian cell without the specialised processing machinery of neuroendocrine cells. The expression constructs are of general use and can be used for expression of any neuropeptide.

In a second aspect, the invention relates to an expression construct for expression in a mammalian cell, the expression construct coding for a heterologous polypeptide comprising at least 80 amino acids, said heterologous polypeptide comprising an N-terminal signal peptide linked to a neuropeptide, the neuropeptide being linked to a C-terminal peptide, wherein the native (wildtype) human cDNA coding for said neuropeptide additionally comprises a sequence coding for a pro-peptide, between the signal peptide and the neuropeptide.

The expression constructs can be used for expression in mammalian producer cells of neuropeptides that normally have a pro-region and a C-terminal peptide. Mammalian producer cells often do not possess the machinery to process the pro-regions of neuropeptides. By deleting the pro-region from the expression construct, correct processing is ensured. In order to secure sufficient length of the expression construct, this aspect is limited to neuropeptides that additionally possess a C-terminal peptide.

In a further aspect the invention relates to an isolated host cell transfected or transduced with the expression construct of the invention.

In a still further aspect the invention relates to a packaging cell line capable of producing an infective virus particle, said virus particle comprising a Retroviridae derived genome comprising a 5′ retroviral LTR, a tRNA binding site, a packaging signal, a promoter operably linked to a polynucleotide sequence comprising the expression construct of the invention, an origin of second strand DNA synthesis, and a 3′ retroviral LTR.

Furthermore the invention relates to an implantable biocompatible cell device, the device comprising:

i) a semipermeable membrane permitting the diffusion of a neuropeptide and/or a virus vector of the invention; and
ii) an inner core comprising a composition of cells according to the invention or a packaging cell line according to any of the invention.

In a further aspect the invention relates to a method of treatment of epilepsy comprising administering to an individual in need thereof a gene therapy vector comprising the expression construct of the invention, a composition of host cells of the invention or a device of the invention.

In a further aspect the invention relates to a method of treatment of Alzheimer's Disease comprising administering to an individual in need thereof a gene therapy vector comprising the expression construct of the invention, a composition of host cells of the invention or a device of the invention.

In a further aspect the invention relates to a method of treatment of Huntington's Disease comprising administering to an individual in need thereof a gene therapy vector comprising the expression construct of the invention, a composition of host cells of the invention or a device of the invention.

In a further aspect the invention relates to a method of treatment of a disease or disorder or damage involving injury to the brain, brain stem, the spinal cord, and/or peripheral nerves, resulting in stroke, traumatic brain injury (TBI), spinal cord injury (SCI), and/or diffuse axonal injury (DAI), said method comprising administering to an individual in need thereof a gene therapy vector comprising the expression construct of the invention, a composition of host cells of the invention or a device of the invention. The injury may be excitotoxic injury.

In a further aspect the invention relates to a method of treatment of a neuropsychiatric disorder. Said neuropsychiatric disorder may be selected from the group consisting of depression, such as medically intractable depression, obsessive compulsory disorder (OCD), Tourette's syndrome, anxiety, bipolar disorders, and phobia comprising administering to an individual in need thereof a gene therapy vector comprising the expression construct of the invention, a composition of host cells of the invention or a device of the invention.

In a further aspect the invention relates to a method of treatment of peripheral neuropathy and/or neuropathic pain comprising administering to an individual in need thereof a gene therapy vector comprising the expression construct of the invention, a composition of host cells of the invention or a device of the invention.

In a further aspect the invention relates to the expression construct of the invention, a composition of host cells of the invention or a device of the invention for use in a method of treatment.

In a further aspect the invention relates to the expression construct of the invention, a composition of host cells of the invention or a device of the invention for use in a method of treatment of epilepsy, Huntington's Disease or Alzheimer's Disease, excitotoxic injury, diseases or disorder or damage involving injury to the brain, brain stem, the spinal cord, and/or peripheral nerves, resulting in stroke, traumatic brain injury (TBI), spinal cord injury (SCI), and/or diffuse axonal injury (DAI), depression, such as medically intractable depression, obsessive compulsory disorder (OCD), Tourette's syndrome, anxiety, bipolar disorders, and phobia peripheral neuropathy, neuropathic pain.

In further aspects the invention relates to a method of treatment of a disorder selected from the group consisting of Huntington's disease, sleeping disorders, narcolepsy and alcoholism, comprising administering to an individual in need thereof a gene therapy vector comprising the expression construct of the invention, a composition of host cells according to the invention or a device of the invention. These indications are based on delivery of orexin peptides.

In further aspects of the invention there is provided a method of treatment of epilepsy, neuropathic pain, peripheral neuropathy, eating disorders, and obesity, comprising administering to an individual in need thereof a gene therapy vector comprising the expression construct of the invention, a composition of host cells according to the invention, or a device of the invention. These aspects are based on delivery of NPY peptides.

### FIGURES

FIG. 1: Expression plasmid for the full length galanin sequence containing an optimal furin recognition sequence in the pro-region. The Flprepro-furin-galanin ORF is placed under transcriptional control of the CA promoter (CMV enhancer, chicken R-actin promoter).

FIG. 2: Expression plasmid for the preproGDNF sequence with an optimal furin recognition sequence fused to the mature galanin sequence. The ppGDNF-furin-galanin ORF is placed under transcriptional control of the CA promoter (CMV enhancer, chicken R-actin promoter).

FIG. 3: Expression plasmid for the mouse immunoglobulin heavy chain V-region signal peptide (IgSP) sequence fused to the delta-prepro galanin sequence. The IgSP-galanin ORF is placed under transcriptional control of the CA promoter (CMV enhancer, chicken β-actin promoter).

FIG. 4: Galanin secretion levels from supernatants of ARPE-19 cells transiently transfected with the pCA expression vectors expressing preproGDNF with a codon-optimized furin recognition sequence fused to the mature galanin sequence (ppGDNF-furin-galanin), mouse immunoglobulin heavy chain V-region signal peptide sequence fused to deltaprepro-galanin (IgSP-galanin) and full length galanin containing a codon-optimized furin recognition sequence in the pro-peptide sequence (FL-prepro-furin-galanin). Supernatants were subjected to galanin ELISA 48 hrs post-transfection.

FIG. 5A: Galanin receptor binding assay. Competition assay, in which the galanin sample competes with ¹²⁵I-galanin for binding to galanin receptor 1 (GalR1). The left figure shows the competition curve for recombinant human mature galanin. The right curve shows supernatant from ARPE-19 cells stably transfected with the FLprepro-furin-galanin expression vector.

FIG. 5B: Galanin receptor binding assay. Competition assay, in which the galanin sample competes with ¹²⁵I-galanin for binding to galanin receptor 1 (GalR1). The left figure shows the competition curve for recombinant human mature galanin. The right curve shows supernatant from ARPE-19 cells stably transfected with the ppGDNF-furin-galanin expression vector.

FIG. 5C: Galanin receptor binding assay. Competition assay, in which the galanin sample competes with ¹²⁵I-galanin for binding to galanin receptor 1 (GalR1). The left figure shows the competition curve for recombinant human mature galanin. The right curve shows supernatant from ARPE-19 cells stably transfected with the IgSP-deltaprepro-galanin expression vector.

FIG. 6: Expression plasmid for the mouse immunoglobulin heavy chain V-region signal peptide (IgSP) sequence fused to the sequence of mature galanin. The IgSP-galanin ORF is placed under transcriptional control of the CMV promoter.

FIG. 7: Expression plasmid for the lymphotoxin signal peptide sequence fused to the sequence of mature galanin. The lymphotoxin-galanin ORF is placed under transcriptional control of the CMV promoter.

FIG. 8: Expression plasmid for the semaphorin signal peptide sequence fused to the sequence of mature galanin. The semaphorin-galanin ORF is placed under transcriptional control of the CMV promoter.

FIG. 9: Galanin secretion levels from supernatants of ARPE-19 cells transiently transfected with the pCl expression vectors expressing lymphotoxin-galanin (Lympho-gala), semaphorin-galanin (Sema-gala), IgSP-galanin (IgSP-gala) and a full length (WT) galanin construct. Supernatants were subjected to galanin ELISA 48 hrs post-transfection.

FIG. 10A: Clustal W sequence alignment of galanin precursor from different species. Pig (SEQ ID NO 27), bovine (SEQ ID NO 28), human (SEQ ID NO 29), rat (SEQ ID NO 30) and mouse (SEQ ID NO 31). Mature galanin peptide is highlighted in bold.

FIG. 10B: Clustal W sequence alignment of mature galanin from different species. Pig (SEQ ID NO 32), bovine (SEQ ID NO 33), human (SEQ ID NO 34), rat (SEQ ID NO 35) and mouse (SEQ ID NO 36).

FIG. 11A: Clustal W alignment of orexin precursor from different species. Rat (SEQ ID NO 37), mouse (SEQ ID NO 38), human (SEQ ID NO 39) and pig (SEQ ID NO 40). Orexin A and B are highlighted in bold.

FIG. 11B: Clustal W alignment of orexin A from different species. Rat (SEQ ID NO 41), mouse (SEQ ID NO 42), human (SEQ ID NO 43) and pig (SEQ ID NO 44).

FIG. 11C: Clustal W alignment of orexin B from different species. Rat (SEQ ID NO 45), mouse (SEQ ID NO 46), human (SEQ ID NO 47) and pig (SEQ ID NO 48).

FIG. 12A: Clustal W alignment of NPY precursor from different species. Rhesus monkey (SEQ ID NO 49), human (SEQ ID NO 50), rat (SEQ ID NO 51) and mouse (SEQ ID NO 52). Mature NPY is highlighted in bold.

FIG. 12B: Clustal W alignment of mature NPY from different species. Rhesus monkey (SEQ ID NO 53), human (SEQ ID NO 54), rat (SEQ ID NO 55) and mouse (SEQ ID NO 56).

FIG. 13A: 2D, 8 week study, Galanin clones. In vitro stable galanin secreting ARPE-19 clones based on the pCA expression vector. The clones were generated using standard transfection techniques and G418 selection. Clones were cultured without passaging for 8 weeks. Galanin secretion was measured by ELISA.

FIG. 13B: 2D, 8 week study, SB IgSP-galanin clones. In vitro stable galanin secreting ARPE-19 clones based on the SB substrate vector pT2. The clones were generated using the SB technology. Clones were cultured without passaging for up to 8 weeks. Galanin secretion was measured by ELISA.

FIG. 14: Galanin 4-week in vivo study in minipigs. Galanin expression levels of encapsulated SB-galanin clone, SB-IgSP-24, made using the SB technology versus clone ppG-152, made using standard transfection techniques. Pre-implantation values (blue bars) are compared to explant values (red bars) and devices run in vitro in parallel (yellow bars). Devices were implanted into the hippocapmus of the minipigs.

### DEFINITIONS

A “neuropeptide” is a member of a class of protein-like molecules expressed in the brain. Neuropeptides consist of short chains of amino acids, with some functioning as neurotransmitters and some functioning as hormones. By short chains are meant peptides with a molecular weight of <5 kDa.

“Introns” refer in this work to those regions of DNA sequence that are transcribed along with the coding sequences (exons) but are then removed in the formation of the mature mRNA. Introns may occur anywhere within a transcribed sequence, between coding sequences of a gene, within the coding sequence of a gene, and within the 5′ untranslated region (5′ UTR) (including the promoter region). Introns in the primary transcript are excised and the exon sequences are simultaneously and precisely ligated to form the mature mRNA. The junctions of introns and exons form the splice sites. The base sequence of an intron conservatively begins with GT and ends with AG in many higher eukaryots.

As used herein “a biocompatible capsule” or “a biocompatible device” means that the device, upon implantation in a host mammal, does not elicit a detrimental host response sufficient to result in the rejection of the device or to render it inoperable, for example through degradation.

As used herein “an immunoisolatory capsule or device” means that the device or capsule upon implantation into a mammalian host minimizes the deleterious effects of the host's immune system on the cells within its core.

Biological activity refers to the biologically useful effects of a molecule on a specific cell. As used herein “a biologically active neuropeptide” is one which is released or secreted from the cell in which it is made and exerts its effect on a target cell. Biological activity of the secreted neuropeptide can be verified by suitable assays, e.g. receptor binding assays such as the GalR1 binding assay described in Example 3 for galanin.

Treatment: “Treatment” can be performed in several different ways, including curative, ameliorating, symptomatic, and as prophylaxis. Curative treatment generally aims at curing a clinical condition, such as a disease or an infection, which is already present in the treated individual. Ameliorating treatment generally means treating in order to improve in an individual an existing clinical condition. Prophylactic treatment generally aims at preventing a clinical condition. Symptomatic treatment generally aims at treating or ameliorating one or more of the symptoms caused by the underlying disease.

By a “mammalian promoter” is intended a promoter capable of functioning in a mammalian cell.

Down regulation of a promoter means the reduction in the expression of the product of transgene to a level which may lead to a lack of significant biological activity of the transgene product after in vivo implantation. As used herein “a promoter not subject to down regulation” means a promoter, which, after in vivo implantation in a mammalian host, drives or continues to drive the expression of transgene at a level which is biologically active.

As used herein “long-term, stable expression of a biologically active neuropeptide” means the continued production of a biologically active neuropeptide at a level sufficient to maintain its useful biological activity for periods greater than one month, preferably greater than three months and most preferably greater than six months.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680)), and the default parameters suggested therein. The ClustalW software is available from as a ClustalW WWW Service at the European Bioinformatics Institute http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similary be used to align nucleotide sequences. Sequence identities may be calculated in a similar way as indicated for amino acid sequences.

### DETAILED DESCRIPTION

The present invention relates to methods and expression constructs ensuring correct processing of neuropeptides expressed recombinantly in non-endocrine cells. Non-endocrine cells lack the complete protein processing apparatus required for the processing of neuropeptides.

Galanin has been used experimentally to illustrate the finding of the present invention. Needless to say, the findings are of general nature and can be applied by the person skilled in the art to other neuropeptides with similar processing requirements.

Galanin (Swissprot accession number P22466) in humans is a 30 amino acid peptide expressed in both the CNS and PNS. Galanin is expressed as a 123 amino acid precursor (FIG. 10) consisting of

Amino acids 1-19 signal peptide
Amino acids 20-30 propeptide
Amino acids 33-62 galanin
Amino acids 65-123 C-terminal peptide or galanin message associated peptide

Following cleavage of the signal peptide, the propeptide is cleaved at the motif PAKEKR↓GW, wherein the arrow marks the cleavage site. The C-terminal peptide is cleaved following the motif: GLTSKR↓EL. Following cleavage of the C-terminal peptide the two C-terminal basic amino acids from the mature peptide, K and R, are trimmed by a carboxypeptidase. The resulting bioactive mature peptide thus consists of amino acids 33-62.

The present inventors initially determined that expression of the wild-type construct in ARPE-19 cells results in secretion of a peptide being active in the GalR1 receptor binding assay. N-terminal sequencing of the purified peptide revealed that the pro-peptide was not cleaved from the mature peptide. Apparently, the ARPE-19 cells do not possess the pro-convertase that can cleave the motif. Therefore, the pro-convertase cleavage site in the pro-peptide of galanin is not an optimal furin site.

An expression construct consisting of GDNF pre-pro region linked to mature galanin also led to secretion of a peptide that could be identified with an anti-galanin antibody. However, a Western Blot revealed that the molecular weight was much higher than expected, corresponding to a peptide consisting of mature galanin and the pro-region of GDNF. This is very surprising in view of the fact that ARPE-19 cells are known to process wild-type GDNF correctly (WO/2007/048413). Evidently, the furin site in the pro-region of GDNF is not an optimal furin-site at least not when linked to a neuropeptide such as galanin.

Other heterologous expression constructs tested were based on long signal peptides (see comparative example 4). It is generally accepted in the art that a certain minimal length is required for a translated peptide to make its way through the secretory pathway of a eukaryotic cell. Therefore a search was made for long signal peptides, which were predicted to lead to correct signal peptide cleavage using the prediction program SignalP. However none of the constructs, ranging in length from 49-68 amino acids, resulted in secretion of galanin.

In view of these observations, the inventors have developed two alternative approaches to recombinant expression of galanin in mammalian cells. Both alternatives provide solutions to the problem of incomplete processing of the pro-peptides.

Thus in one aspect the invention relates to an expression construct for expression in a mammalian cell, the expression construct coding for a heterologous polypeptide comprising at least 51 amino acids, said heterologous polypeptide comprising from the N-terminal to the C-terminal a mammalian signal peptide, a pro-peptide, and a neuropeptide, wherein said neuropeptide is cleavable from the pro-peptide by furin at a furin-site, and wherein said furin-site is optimal for cleavage.

By optimising the pro-convertase cleavage site of e.g. galanin and GDNF correct processing in ARPE-19 cells has been made possible. In one embodiment, this has been done without mutating the neuropeptide.

In another aspect, the invention relates to an expression construct for expression in a mammalian cell, the expression construct coding for a heterologous polypeptide comprising at least 80 amino acids, said heterologous polypeptide comprising an N-terminal signal peptide linked to a neuropeptide, the neuropeptide being linked to a C-terminal peptide, wherein the native (wildtype) human cDNA coding for said neuropeptide additionally comprises a sequence coding for a pro-peptide, between the signal peptide and the neuropeptide.

According to this embodiment, the pro-region of e.g. galanin has been deleted to create a so-called deltapro-construct. The C-terminal peptide of the neuropeptide is maintained in order to secure sufficient length of the construct. Surprisingly, the non-endocrine ARPE-19 cells have no problems cleaving the C-terminal peptide from the mature peptide. It is also unexpected that the neuropeptides can assume the correct biologically active conformation without the pro-region, which is often implicated in this process.

While it is stated in the present invention that certain parts of the encoded polypeptides are linked to one-another, this does not exclude the possibility that a non-functional stretch of amino acids is inserted e.g. between signal peptide and pro-peptide; between signal peptide and mature peptide, between pro-peptide and mature peptide, between mature peptide and C-terminal peptide. Examples of such non-functional stretch of amino acids include a peptide tag, sorting signals, albumin, a transmembrane region. This could be done without departing from the teaching of the invention as long as it is ensured that the signal peptide, the pro-peptide, and the C-terminal peptide can be cleaved and that the secreted neuropeptide is bioactive. It is also conceivable that other stretches of amino acids could be fused to the C-terminal of bioactive neuropeptide. It should be understood that in a preferred embodiment, the encoded polypeptide does not comprise such non-functional stretch or stretches of amino acids.

The coding sequences inserted into the expression vectors of the present invention may be codon-optimised for expression in a host cell. For example they may be codon optimised for expression in eukaryotes, in mammals, or preferably in human beings.

In general, any of the nucleic acids of the invention can be modified to increase expression in a particular host, using the generally know techniques for codon optimization. Codons that are utilized most often in a particular host are called optimal codons, and those not utilized very often are classified as rare or low-usage codons. Codons can be substituted to reflect the preferred codon usage of the host, a process called “codon optimization”. Optimized coding sequences comprising codons preferred by a particular prokaryotic or eukaryotic host can be used to increase the rate of translation or to produce recombinant RNA transcripts having desirable properties, such as a longer half-life, as compared with transcripts produced from a non-optimized sequence. Techniques for producing codon optimized sequences are known generally in the art.

### Pre-Pro Regions

In the first aspect of the invention the expression construct comprises a pre-pro-peptide with an optimal furin site. The following section concerning pre-pro-peptides concerns this aspect.

The length of the heterologous polypeptide including signal peptide, pro-region with optimal furin site, the neuropeptide and any optional C-terminal peptide, preferably is at least 55 amino acids, such as at least 60 amino acids, for example at least 65 amino acids, such as at least 70 amino acids, for example at least 75 amino acids, such as at least 80 amino acids, for example at least 90 amino acids, such as at least 100, for example at least 110, such as at least 120, for example at least 130, such as at least 140, for example at least 150, such as at least 160, for example at least 170, such as at least 180, for example at least 190, such as at least 200 amino acids.

Preferably, the heterologous polypeptide comprises less than 200 amino acids, such as less than 190, for example less than 180, such as less than 170, for example less than 160, preferably less than 150, for example less than 140, more preferably less than 130, for example less than 120, such as less than 110 amino acids.

The experiments with the long signal peptides have shown that the total length of the encoded polypeptide is not the only decisive factor for successful secretion. The present inventors believe that the length of the encoded polypeptide after cleavage of the signal peptide is an important factor for successful secretion. Thus the 30 amino acids of mature galanin may be too short a peptide for it to be secreted from a mammalian cell. Therefore, preferably the length of the heterologous polypeptide following cleavage of the signal peptide, i.e. the length of the pro-region, the neuropeptide and any optional C-terminal peptide is preferably at least 35 amino acids, preferably at least 40 amino acids, such as at least 50 amino acids, for example at least 60 amino acids, such as at least 70 amino acids, for example at least 80 amino acids, such as at least 90 amino acids, for example at least 100 amino acids.

In embodiments of the invention the heterologous polypeptide comprises from 51 to 150 amino acids, such as from 55 to 150 amino acids, for example from 60 to 150 amino acids, such as from 70 to 150 amino acids, for example from 80-150 amino acids, such as 90-130 amino acids, for example 90-120 amino acids, such as 90-110 amino acids, or 100-120 amino acids.

The pre-pro-region may originate from the same cDNA as the neuropeptide or from a cDNA that is heterologous with respect to the neuropeptide.

The signal peptide and the pro-peptide comprising the optimal furin site may be derived from the same pre-pro-protein precursor. For example the precursor protein may be galanin. Alternatively, the precursor protein may be a neurotrophic factor.

Suitable neurotrophic factors, the pre-pro-regions of which may be used include but is not limited to a GDNF family protein, such as GDNF, Neublastin, Neurturin, and persephin, preferably GDNF. Other suitable neurotrophic factor include neurotrophins, such as BDNF, NT4-5, NT3, and NGF, preferably NGF.

Other suitable precursor proteins to make up the pre- and pro-regions include proteins selected from the group consisting of amphiregulin, transforming growth factor-beta1, von Willebrand factor, furin, Kex2, PACE, subtilisin.

In a preferred embodiment of the invention the pre-pro-region has at least 70% sequence identity to a wild-type pre-pro-region, more preferably at least 75%, such as at least 80%, for example at least 85%, such as at least 90%, for example at least 95%, such as at least 98% sequence identity.

### Furin-Sites

In the first aspect of the invention the expression construct comprises a pre-pro-peptide with an optimal furin site. The following section concerning furin-sites concerns this aspect.

Furin sites have been described extensively in Duckert et al (Protein Engineering, Design, and Selection, Vol 17, No. 1, 170-112, 2004). According to Duckert et al, a furin consensus sequence is R-X-K/R-R↓, wherein cleavage takes place at the arrow. In a study of furin sites in 38 proteins, it turned out that 31 of the 38 proteins had this motif but other motifs were also seen. The following general rules for furin specificity were proposed:
(i) an arginine at P1 is essential for cleavage
(ii) in addition to the P1 arginine, at least two out of P2, P4 and P6 are required to be basic for efficient cleavage, and
(iii) at the P1′ position an amino acid residue with a hydrophobic aliphatic side chain (i.e. leucine, isoleucine, or valine) is not suitable.

According to these rules, the pro-protein convertase site in human galanin PAKEKR↓GW is a furin site. So is the pro-protein convertase site in human GDNF pre-pro peptide fused to human galanin IKRLKR↓GW. However, expression of these constructs in ARPE-19 cells did not lead to processing of the furin sites. Therefore, these sites cannot be regarded as “optimal furin sites” in accordance with the present invention.

A preferred optimal furin site according to the present invention comprises the following amino acid sequence: P6P5P4P3P2P1↓P1′P2′, wherein cleavage takes place at ↓ between P1 and P1′, and wherein P6P5P4P3P2P1P1′P2′ is R-X-R-X-[K/R]-R↓X-X, wherein X is any amino acid.

Preferably, P1′ is neither isoleucine (I), leucine (L) nor valine (V). P2′ is preferably neither arginine (R) nor lysine (K).

More preferably, P6P5P4P3P2P1P1′P2′ of the furin site is R-X-R-X-K-R↓X-X. For example P6P5P4P3P2P1P1′P2′ may be R-X-R-K-K-R↓X-X or R-X-R-T-K-R↓X-X.

Preferably P1′ is not S. In another preferred embodiment, P2′ is neither S nor P.

### Signal Peptides

A eukaryotic signal peptide is a peptide present on proteins that are destined either to be secreted or to be membrane components. It is usually N-terminal to the mature bioactive protein. In the present context, all signal peptides identified in the program SignalP (version 3.0. Improved prediction of signal peptides: SignalP 3.0. Jannick Dyrløv Bendtsen, Henrik Nielsen, Gunnar von Heijne and Søren Brunak. J. Mol. Biol., 340:783-795, 2004.) are considered signal peptides.

The targeting of secreted and proteins to the secretory pathway is accomplished via the attachment of a short, amino-terminal sequence, known as the signal peptide or signal sequence (von Heijne, G. (1985) J. Mol. Biol. 184, 99-105; Kaiser, C. A. & Botstein, D. (1986), Mol. Cell. Biol. 6, 2382-2391). The signal peptide itself contains several elements necessary for optimal function, the most important of which is a hydrophobic component. Immediately preceding the hydrophobic sequence is often a basic amino acid or acids, whereas at the carboxyl-terminal end of the signal peptide are a pair of small, uncharged amino acids separated by a single intervening amino acid which defines the signal peptidase cleavage site.

A preferred mammalian signal peptide is from 15 to 30 amino acids long (average for eukaryotes is 23 amino acids). The common structure of signal peptides from various proteins is commonly described as a positively charged n-region, followed by a hydrophobic h-region and a neutral but polar c-region. The (−3,−1)-rule states that the residues at positions −3 and −1 (relative to the cleavage site) must be small and neutral for cleavage to occur correctly.

The n-region of eukaryotic signal sequences is only slightly Arg rich. The h-region is short and very hydrophobic. The c-region is short and has no observable pattern. As described the −3 and −1 positions consist of small and neutral residues. The amino acid residues C-terminal to the cleavage site is of less importance in eukaryotes.

In the C-region the residues at position −1 and −3 are the most important. These are small, uncharged amino acids. At position −1 the residue is preferably A, G, S, I, T or C. More preferably the −1 position is A, G or S. At position −3 the residue is preferably A, V, S, T, G, C, I, or D. More preferably, the −3 position is A, V, S or T.

The hydrophobic region prevalently consists of hydrophobic residues. These include A, I, L, F, V, and M. Preferably, at positions −6 to −13. Of the 8 amino acids constituting this region, at least 4 residues should be hydrophobic, more preferably at least 5, more preferably at least 6, such as 7 or 8.

During the secretion process, the signal peptide of the pre-pro-protein or pre-protein is cleaved by the host cell expressing the neuropeptide. While the cleavage site is generally defined, a skilled artisan will appreciate that there can be variability in the signal peptide cleavage site. Accordingly, embodiments having some ambiguity with respect to the exact cleavage site are within the scope of the invention.

The signal peptide may be any functional signal peptide, such as a heterologous signal peptide, such as a mammalian signal peptide. The signal peptide may be from any suitable species, such as human, mouse, rat, monkey, pig, dog, cat, cow or horse.

The signal peptide in the first aspect is linked to the pro-region. According to the second aspect it is directly fused to said neuropepetide, such as the C-terminal end of the signal peptide being fused to the N-terminal end of the neuropeptide.

The inventors have discovered that heterologous signal peptides are useful and often provide a higher yield than the native signal peptide.

Preferably the signal peptide is a mammalian signal peptide. For example the signal peptide may be a human signal peptide, a rat signal peptide, a mouse signal peptide, a porcine signal peptide, a simian signal peptide, a canine signal peptide, a feline signal peptide, a bovine signal peptide, or an equine signal peptide.

The heterologous signal peptide can be selected from the group consisting of a growth factor signal peptide, a hormone signal peptide, a cytokine signal peptide and an immunoglobulin signal peptide.

Thus, examples of signal peptides are signal peptides selected from the group consisting of TGFβ signal peptides, GDF signal peptides, IGF signal peptides, BMP signal peptides, Neurotrophin signal peptides, PDGF signal peptide and EGF signal peptide, signal peptides selected from a hormone signal peptide, said hormone being selected from the group consisting of growth hormone, insulin, ADH, LH, FSH, ACTH, MSH, TSH, T3, T4, and DHEA, or an interleukin signal peptide.

In one embodiment, the signal peptide is selected from the group consisting of neurturin signal peptide, GDNF signal peptide, persephin signal peptide, and NGF signal peptide.

In another embodiment, the signal peptide is selected from the group consisting of albumin signal peptide, modified albumin signal peptide, and growth hormone signal peptide, such as a signal peptide selected from the group consisting of rat albumin signal peptide, and human growth hormone signal peptide, such as rat albumin signal peptide and human growth hormone signal peptide. These signal peptides are known from WO 2004/108760 (NsGene & Biogen Idec).

Thus, in some embodiments, the signal peptide is a native rat albumin signal peptide. In other embodiments, the signal peptide is a human growth hormone signal peptide.

(SEQ ID NO 19; Human Growth hormone SP)
MATGSRTSLLLAFGLLCLSWLQEGSA
(SEQ ID NO 20; Albumin SP)
MKWVTFLLLLFISGSAFS
(SEQ ID NO 21; Modified Albumin SP)
MKWVTFLLFLLFISGDAFA

In yet another embodiment, the signal peptide is an immunoglobulin signal peptide, such as the immunoglobulin heavy chain signal peptide. In particular, an immunoglobulin signal peptide may be a signal peptide selected from the group consisting of mouse IgSP (SEQ ID NO 16), rat IgSP (SEQ ID NO 18), porcine IgSP (SEQ ID NO 17 simian IgSP (SEQ ID NO 14 or 15), human IgSP (SEQ ID NO 13), such as mouse IgSP (SEQ ID NO 16) or human IgSP (SEQ ID NO 13).

### Exemplary IgSPs

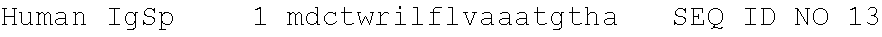

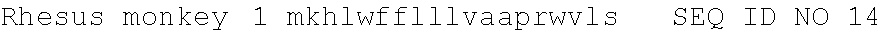

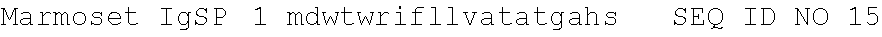

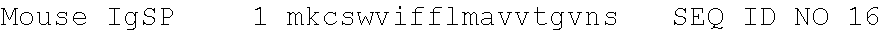

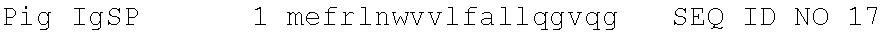

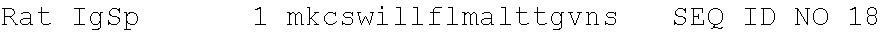

Immunoglobulin signal peptide (IgSP) is a small 19 amino acid peptide known from a large group of mammals. The sequences from human, rhesus monkey, marmoset, rat, mouse and pig are aligned in above. The percent sequence identity compared to human IgSP varies from 21 (pig) to 68 (marmoset) percent. This relatively large variation indicates that the specific sequence can be altered to a large extent without substantially changing the biological function of the signal peptide.

Preferably the IgSP is of mouse or human origin because the mouse IgSP is known to be functional in mouse, rat and human beings.

In another embodiment the signal peptide is a native neuropeptide signal peptide such as a native human galanin signal peptide.

### Neuropeptides

A “neuropeptide” is a member of a class of protein-like molecules expressed in the brain. Neuropeptides consist of short chains of amino acids, with some functioning as neurotransmitters and some functioning as hormones. By short chains are meant peptides with a molecular weight of <5 kD.

The present invention relates to expression in mammalian cells of relatively small peptides. Such peptides are inherently difficult to express, process and secrete from mammalian cells due to their special processing sites and small size. In embodiments of the invention the neuropeptide comprises less than 50 amino acids, more preferably less than 40 amino acids, more preferably less than 35 amino acids, such as less than 30 amino acids, for example less than 25 amino acids, such as less than 20 amino acids, for example less than 15 amino acids, such as less than 10 amino acids.

Preferably the neuropeptide comprises between 10 and 50 amino acids, such as between 15 and 40, for example between 20 and 30 amino acids.

Preferably the neuropeptide is a human neuropeptide.

According to the first aspect of the invention the neuropeptide may be selected from the group consisting of galanin, neuropeptide Y, orexin A, orexin B, enkephalin, somatostatin 14, somatostatin 28, vasoactive intestinal peptide, intestinal peptide PHV-42, intestinal peptide PHV-27, substance P, neurotensin, cholecystokinin 58, cholecystokinin 39, cholecystokinin 33, cholecystokinin 25, cholecystokinin 18, cholecystokinin 12, cholecystokinin 8, cholecystokinin 7, cholecystokinin 5, substance P, neuropeptide K, neuropeptide gamma, neurokinin A, TRH. All of these small peptides can be linked to the pre-pro regions with optimal furin site described in the first aspect of the invention. This ensures that the length of the expression construct is sufficient to result in secretion and that its processing can be carried out by expressor cells without the specialised processing machinery of neuroendocrine cells. In a preferred embodiment, the neuropeptide is selected from the group consisting of galanin, neuropeptide Y, orexin A, and orexin B.

In a particularly preferred embodiment the heterologous polypeptide comprises GDNF pre-pro-region with an optimal furin site, linked to galanin such as the sequence shown in SEQ ID No 4.

In another particularly preferred embodiment the heterologous polypeptide comprises galanin pre-pro-region with an optimal furin site, linked to galanin mature peptide and including galanin C-terminal peptide such as the sequence shown in SEQ ID No 2.

According to the second aspect of the invention, relating to expression constructs without a pro-region, the neuropeptide may be selected from the group consisting of galanin, cholocystokinin, Neurotensin, substance P, neuropeptide K, neutopeptide gamma, Neurokinin A, vasoactive intestinal peptide, and orexin-B. Preferably the neuropeptide is galanin.

### Preferred Neuropeptides

### Galanin

Galanin is a small highly conserved peptide of 29 amino acids in animals and 30 amino acids in humans (FIG. 10). The galanin precursor (FIG. 10A) consists of a signal peptide, a pro-peptide, galanin peptide and a C-terminal peptide also known as galanin message associated peptide.

Galanin is produced in the CNS and PNS and has a widespread distribution in the brain. This is consistent with findings that galanin regulates diverse functions such as learning, memory, mood, feeding behaviour, and pain perception. The antiepileptic effects of galanin have been shown in various animal models. The first report on galanin's anticonvulsive effect in rat brains came in 1992 (Mazarati et al, 1992, Brain Res 589:164-66). The effect was most prominent when galanin was injected into the hippocampus of rats with chemically induced status epilepticus (Mazarati et al, op cit). Later it has been shown that galanin also exhibits neuroprotective effect on hippocampal neurons (Habermann et al, 2003, Nat Med 9, 1076-80; Elliott-Hunt et al, 2004, PNAS, 101:5105-10), which also express 2 of 3 galanin receptors—GalR1 and GalR2 (Mazarati et al, 2004, Neuropeptides, 38:331-43). In experiments with wild type, galanin knockout, and galanin overexpressing mice, seizures more readily generated in galanin knockout mice and less readily in galanin overexpressing mice as compared to wild type controls (Mazarati et al, 200, J NeuroSci, 20:6276-81). In addition, gene therapy using AAV vectors expressing galanin was effective in animal models of focal epilepsy.

In a preferred embodiment, the galanin peptide of the invention comprises all the residues marked in mature galanin as fully conserved in the alignment in FIG. 10B. More preferably, the galanin neuropeptide is human mature galanin peptide. It is to be understood that the galanin peptides of the invention are biologically active for example by being able to bind to the galanin receptor 1 in the receptor binding assay described in the examples.

Preferably for the C-terminal peptide, the galanin peptide comprises the residues of the C-terminal peptide marked in the alignment of FIG. 10B as fully conserved. More preferably, the C-terminal peptide is the human C-terminal galanin peptide.

### Orexins

Orexins, also called hypocretins, are the common names given to a pair of highly excitatory neuropeptide hormones. The two related peptides (Orexin-A and B, or hypocretin-1 and -2), with approximately 50% sequence identity, are produced by cleavage of a single precursor protein. Orexin-A/hypocretin-1 consists of 33 amino acid residues and has two intrachain disulfide bonds, while Orexin-B/hypocretin-2 is a linear 28 amino acid residue peptide. Studies suggest that orexin A/hypocretin-1 may be of greater biological importance than orexin B/hypocretin-2. Although these peptides are produced by a very small population of cells in the lateral and posterior hypothalamus, they send projections throughout the brain. The orexin peptides bind to the orexin receptor, a G-protein coupled receptor.

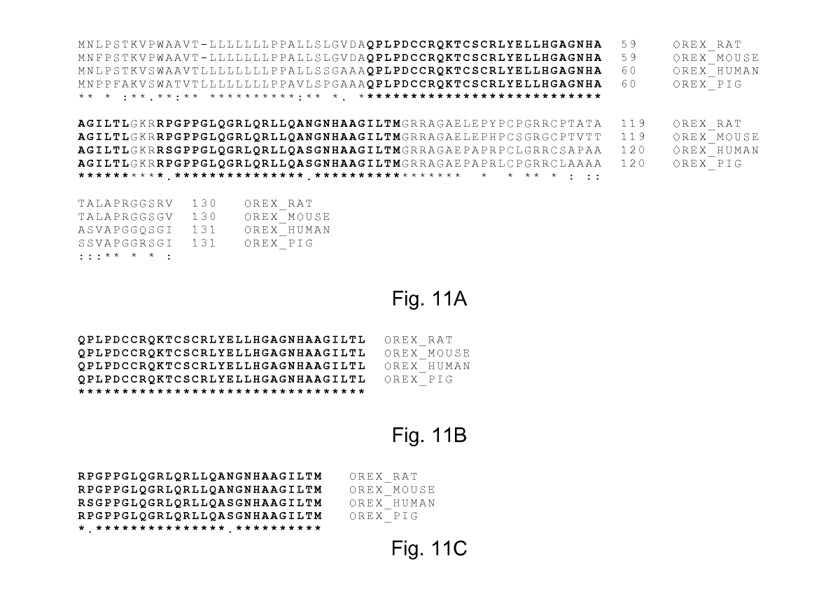

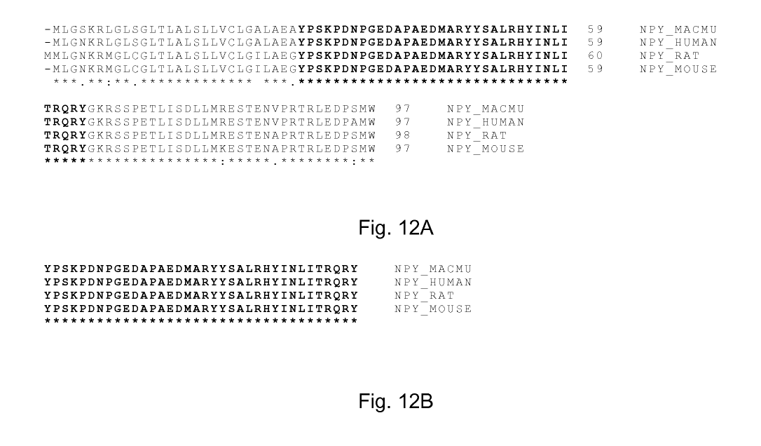

The Orexin precursor (FIG. 11A) consists of a signal peptide, Orexin A and Orexin B, and a C-terminal pro-peptide.

The orexins/hypocretins are strongly conserved peptides, found in all major classes of vertebrates. The peptides are thought to have arisen early in vertebrate evolution.

Orexin seems to promote wakefulness. The discovery that orexin/hypocretin dysregulation causes the sleep disorder narcolepsy (Siegel, 1999, Cell 98:409-12) in mice subsequently indicated a major role for this system in sleep regulation. Narcolepsy results in excessive daytime sleepiness, inability to consolidate wakefulness in the day (and sleep at night), and cataplexy (loss of muscle tone in response to strong, usually positive, emotions). Dogs that lack a functional receptor for orexin/hypocretin have narcolepsy, while animals and people lacking the orexin/hypocretin neuropeptide itself also have narcolepsy. Orexin/hypocretin neurons strongly excite various brain nuclei with important roles in wakefulness including the dopamine, norepinephrine, histamine and acetylcholine systems and appear to play an important role in stabilizing wakefulness and sleep.

Recent studies indicate that a major role of the orexin/hypocretin system is to integrate metabolic, circadian and sleep debt influences to determine whether the animal should be asleep or awake and active. Central administration of orexin A/hypocretin-1 strongly promotes wakefulness, increases body temperature, locomotion and elicits a strong increase in energy expenditure. Sleep deprivation also increases orexin A/hypocretin-1 transmission. The orexin/hypocretin system may thus be more important in the regulation of energy expenditure than food intake. In fact, orexin/hypocretin-deficient narcoleptic patients have increased obesity rather than decreased BMI, as would be expected if orexin/hypocretin were primarily an appetite stimulating peptide. Another indication that deficits of orexin cause narcolepsy is that depriving monkeys of sleep for 30-36 hours and then injecting them with the neurochemical alleviates the cognitive deficiencies normally seen with such amount of sleep loss (Deadwyler et al, 2007, J NeuroSci 27: 14239-47).

Recently, orexin-knock-out transgenic mice have been generated. The knock-out mice transition frequently and rapidly between sleep and wakefulness, displaying many of the symptoms of narcolepsy. The knock-out mice may be used to as an animal model narcolepsy to study the disease and further strengthens the association between orexin and narcolepsy (Mochizuki et al, 2004, J NeuroSci, 24:6291-300).

Preliminary research has been conducted that shows potential for orexin blockers in the treatment of alcoholism. Lab rats given drugs which targeted the orexin system lost interest in alcohol despite being given free access in experiments (Lawrence et al, 2006, Br J Pharmacol 148:752-9).

Furthermore, it has been shown that a mouse model of Huntingtons Disease (HD) shows a dramatic atrophy and loss of orexin neurons in the lateral hypothalamus of R6/2 mice. Importantly, there was also found a significant atrophy and loss of orexin neurons in Huntington patients (Petersen et al, 2005, Hum Mol. Genet. 2005 Jan. 1; 14(1):39-47. Epub 2004 Nov. 3).

Finally, a recent study reported that transplantation of orexin/hypocretin neurons into the pontine reticular formation in rats is feasible, indicating the development of alternative therapeutic strategies in addition to pharmacological interventions to treat narcolepsy (Arias-Carrion 2004, Sleep, 27:1465-70).

In conclusion, the present inventors contemplate the use of orexin expressing cell lines and/or gene therapy vectors in the treatment of Huntington's disease, sleeping disorders, narcolepsy, and/or alcoholism.

The Orexin A and B neuropeptides of the present invention preferably comprise the residues marked as fully conserved in the alignment in FIG. 11B or C. More preferably, the Orexin A and B neuropeptides are human Orexin A and human Orexin B. It is to be understood that the Orexin peptides of the invention are biologically active for example by being able to bind to and activate the same receptor as human Orexin.

### NPY

Neuropeptide Y (NPY) is a 36 amino acid peptide neurotransmitter found in the brain and autonomic nervous system. NPY is expressed as a precursor consisting of (FIG. 12) a signal peptide, the mature NPY peptide, and a C-terminal pro-peptide.

NPY has been associated with a number of physiological processes in the brain, including the regulation of energy balance, memory and learning, and epilepsy. The main effect is increased food intake and decreased physical activity. NPY is secreted by the hypothalamus, and in addition to increasing food intake, it increases the proportion of energy stored as fat and blocks nociceptive signals to the brain. NPY also augments the vasoconstrictor effects of noradrenergic neurons.

NPY has been tested several times in animal models of epilepsy (WO 03/093295; Sørensen et al, Hippocampal NPY gene transfer attenuates seizures without affecting epilepsy-induced impairment of LTP. Exp Neurol. 2008 Nov. 10. [Epub ahead of print]).

NPY has been implicated in symptoms and treatment of neuropathic pain and peripheral neuropathy (Neuropeptide Y acts at Y1 receptors in the rostral ventral medulla to inhibit neuropathic pain. Taylor B K et al, Pain. 2007 September; 131(1-2):83-95; NPY and pain as seen from the histochemical side. Hökfelt T et al, Peptides. 2007 Feb.; 28(2):365-72. Epub 2007 Jan. 17. Review.).

In conclusion, the present inventors contemplate the use of NPY expressing cell lines and/or gene therapy vectors in the treatment of epilepsy, neuropathic pain, peripheral neuropathy, eating disorders, and obesity.

The NPY of the present invention preferably comprises the residues marked as fully conserved in the alignment in FIG. 12B. More preferably, NPY is human NPY. It is to be understood that the NPY peptides of the invention are biologically active for example by being able to bind to and activate the same receptor as human NPY.

### Cell Lines

In one aspect the invention relates to isolated host cells genetically modified with the vector according to the invention.

According to one embodiment, the host cells are eukaryotic producer cells from non-mammals, including but not limited to known producer cells such as yeast (Saccharomyces cerevisiae, and S. pombe), filamentous fungi such as aspergillus, and insect cells, such as Sf9.

According to another embodiment, the cells preferably are mammalian host cells. Preferred species include the group consisting of human, feline, porcine, simian, canina, murine, rat, rabbit, mouse, and hamster.

Examples of primary cultures and cell lines that are good candidates for transduction or transfection with the vectors of the present invention include the group consisting of CHO, CHO-K1, HEI193T, HEK293, COS, PC12, HiB5, RN33b, neuronal cells, foetal cells, ARPE-19, C2C12, HeLa, HepG2, striatal cells, neurons, astrocytes, and interneurons. Preferred cell lines for mammalian recombinant production include CHO, CHO-1, HEI193T, HEK293, COS, PC12, HiB5, RN33b, and BHK cells.

For ex vivo gene therapy, the preferred group of cells includes neuronal cells, neuronal precursor cells, neuronal progenitor cells, stem cells and foetal cells.

The invention also relates to cells suitable for biodelivery of a neuropeptide via naked or encapsulated cells, which are genetically modified to overexpress a neuropeptide, and which can be transplanted to the patient to deliver bioactive neuropeptide locally. Such cells may broadly be referred to as therapeutic cells.

For biodelivery, the host cell is preferably selected from the group consisting of immortalised retinal pigmented epithelial cells, such as ARPE-19 cells, immortalised human fibroblasts, and immortalised human astrocytes.

In one embodiment, the cells are not derived from a human embryo.

The cells may be attached to a matrix.

Cells suitable for naked biodelivery (ex vivo gene therapy) may be selected from the group consisting of stem cells, including human neural stem or precursor cells, human glial stem or precursor cells, and foetal stem cells.

The invention relates to neuropeptide-secreting human cell lines, which have been immortalised by insertion of a heterologous immortalisation gene; to cell lines that are spontaneously immortal; and to growth factor expanded cell lines. In a preferred embodiment of the invention, the human cell line has not been immortalised with the insertion of a heterologous immortalisation gene. As the invention relates to cells which are particularly suited for cell transplantation, preferably as encapsulated cells, such immortalised cell lines are less preferred as there is an inherent risk that they start proliferating in an uncontrolled manner inside the human body and potentially form tumours if they carry known oncogenes.

Growth factor expanded cell lines have the advantage that they depend on added mitogens for continued proliferation. Therefore upon withdrawal of the mitogen prior to or in connection with the filling of a device with cells, the cells stop proliferating and will not proliferate again after implantation into the human body. Some growth factor expanded cell lines may also differentiate upon withdrawal of the mitogen. Growth factor expanded cell lines include stem cells, such as neural stem cells and embryonal stem cells.

Preferably, the cell line is capable of phagocytising. Through phagocytosis the cells will be capable of clearing debris shed by decaying or dying cells within the device.

Preferably, the cell line is a contact inhibited cell line. By a contact inhibited cell line is intended a cell line which when grown in culture flasks as a monolayer under conventional conditions grows to confluency and then substantially stops dividing. This does not exclude the possibility that a limited number of cells escape the monolayer. Inside a capsule or device, the cells grow to confluency and then significantly slow down proliferation rate or completely stop dividing.

A particularly preferred type of cells include epithelial cells which are by their nature contact inhibited and which form stable monolayers in culture.

Even more preferred are retinal pigment epithelial cells (RPE cells). The source of RPE cells is by primary cell isolation from the mammalian retina. RPE cells are capable of phagocytising and are also contact-inhibited cells.

Protocols for harvesting RPE cells are well-defined (Li and Turner, 1988, Exp. Eye Res. 47:911-917; Lopez et al., 1989, Invest. Ophthalmol. Vis. Sci. 30:586-588) and considered a routine methodology. In most of the published reports of RPE cell cotransplantation, cells are derived from the rat (Li and Turner, 1988; Lopez et al., 1989). According to the present invention RPE cells are derived from humans. In addition to isolated primary RPE cells, cultured human RPE cell lines may be used in the practice of the invention.

All normal diploid vertebrate cells have a limited capacity to proliferate, a phenomenon that has come to be known as the Hayflick limit or replicative senescence. In human fibroblasts, this limit occurs after 50-80 population doublings, after which the cells remain in a viable but non-dividing senescent state for many months. This contrasts to the behavior of most cancer cells, which have escaped from the controls limiting their proliferative capacity and are effectively immortal.

It is preferable that the cells are capable of undergoing a certain number of cell divisions so they can be genetically modified and expanded to produce enough cells for encapsulated cell therapy or transplantation therapy. Accordingly a preferred cell line is capable of undergoing at least 50 doublings, more preferably at least 60 doublings, more preferably at least 70 doublings, more preferably at least 80 doublings, more preferably at least 90 doublings, such as approximately 100 doublings.

For encapsulation, the cells need to be able to survive and maintain a functional neuropeptide secretion at the low oxygen tension levels of the CNS. Preferably the cell line of the invention is capable of surviving at an oxygen tension below 5%, more preferably below 2%, more preferably below 1%. 1% oxygen tension corresponds to the oxygen level in the brain.

To be a platform cell line for an encapsulated cell based delivery system, the cell line should have as many of the following characteristics as possible: (1) The cells should be hardy, i.e. viable under stringent conditions (the encapsulated cells should be functional in the vascular and avascular tissue cavities such as in the central nervous system intraparenchymally or within the ventricular or intrathecal fluid spaces or the eye, especially in the intra-ocular environment). (2) The cells should be able to be genetically modified to express neuropeptide. (3) The cells should have a relatively long life span (the cells should produce sufficient progenies to be banked, characterised, engineered, safety tested and clinical lot manufactured). (4) The cells must be of human origin (which increases compatibility between the encapsulated cells and the host). (5) The cells should exhibit greater than 80% viability for a period of more than one month in vivo in the device (which ensures long-term delivery). (6) The encapsulated cells should deliver an efficacious quantity of neuropeptide (which ensures effectiveness of the treatment). (7) When encapsulated, the cells should not cause a significant host immune reaction (which ensures the longevity of the graft). (8) The cells should be non-tumourigenic (to provide added safety to the host, in case of device leakage).

In a screening and characterisation of several cell lines it has been found that the ARPE-19 cell line (Dunn et al., 62 Exp. Eye Res. 155-69 (1996), Dunn et al., 39 Invest. Ophthalmol. Vis. Sci. 2744-9 (1998), Finnemann et al., 94 Proc. Natl. Acad. Sci. USA 12932-7 (1997), Handa et al., 66 Exp. Eye. 411-9 (1998), Holtkamp et al., 112 Clin. Exp. Immunol. 34-43 (1998), Maidji et al., 70 J. Virol. 8402-10 (1996)) has all of the characteristics of a successful platform cell for an encapsulated cell-based delivery system (U.S. Pat. No. 6,361,771, Tao et al). The ARPE-19 cell line was superior to the other cell lines tested.

The ARPE-19 cell line is available from the American Type Culture Collection (ATCC Number CRL-2302). The ARPE-19 cell line is derived from cultures of normal retinal pigmented epithelial (RPE) cells and expresses the retinal pigmentary epithelial cell-specific markers CRALBP and RPE-65. ARPE-19 cells form stable monolayers, which exhibit morphological and functional polarity.

ARPE-19 cells may be cultured in Complete Growth Medium, the serum-containing medium recommended by the cell depositor. Complete Growth Medium is either a 1:1 mixture of Dulbecco's modified Eagle's medium and Ham's F12 medium with 3 mM L-glutamine, 90%; foetal bovine serum, 10% or a 1:1 mixture of Dulbecco's modified Eagle's medium and Ham's F12 medium with HEPES buffer containing 10% fetal bovine serum, 56 mM final concentration sodium bicarbonate and 2 mM L-glutamine. The cells are preferably incubated at 37° C. in 5% CO₂. The cells are typically plated and grown in Falcon tissue culture treated 6 or 12-well plates or T25 or T75 flasks.

For subculturing, medium is removed, and the ARPE-19 cells are preferably rinsed with 0.05% trypsin, 0.02% EDTA solution, and the trypsin is removed. One to two ml of additional trypsin solution is added. The culture is incubated at room temperature (or at 37° C.) until the ARPE-19 cells detach. A subcultivation ratio of 1:3 to 1:5 is recommended.

The hardiness of candidate cell lines for encapsulated cell therapy can be tested using the following three-step screen. (a) Cell viability screen (The cells may be evaluated under stressed conditions using artificial aqueous humor (aAH) medium or artificial cerebral spinal fluid (aCSF) medium). (b) In vitro ECM screen (The cells may be evaluated in an in vitro extra-cellular matrix (ECM) screen). (c) In vivo device viability screen (The encapsulated cells may be evaluated in an in vivo membrane screen). A detailed description of the screens and results with several human and non human cell lines are found in U.S. Pat. No. 6,361,771.

In the three types of screens described above, ARPE-19 cells has proven superior to a number of other cell lines tested (see U.S. Pat. No. 6,361,771).

In another embodiment the cell line is selected from the group consisting of: human immortalised fibroblast cell lines, human immortalised mesencymal stem cell lines, human immortalised astrocyte cell lines, human immortalised mesencephalic cell lines, and human immortalised endothelial cell lines, preferably immortalised with SV40T, vmyc, or the catalytic subunit of telomerase (TERT).

Another type of preferred human cells according to the invention are immortalised human astrocyte cell lines. These cell lines may also have the properties required for the uses according to the present invention. The method for generating an immortalised human astrocyte cell lines has previously been described (Price T N, Burke J F, Mayne L V. A novel human astrocyte cell line (A735) with astrocyte-specific neurotransmitter function. In Vitro Cell Dev Biol Anim. 1999 May; 35(5):279-88.). This protocol may be used to generate astrocyte cell lines.

A further type of preferred cell lines for encapsulated cell biodelivery is choroid plexus cells, of mammalian, preferably murine, more preferably human origin.

In order to generate monoclonal cell lines, cells that have been genetically modified to secrete neuropeptide are seeded under conditions allowing only survival of transfected cells as described in Example 1. After selection of surviving cells or colonies, these may be expanded to form compositions of monoclonal cell lines. Generation of monoclonal cell lines can also be generated using limited dilution, which method requires test of every single selected clone, as there is no selection of transfected cells, or by using single cell sorting.

The monoclonal cell lines can subsequently be subjected to selection for high secretion of neuropeptide, to in vitro and in vivo long term stability screening, before a suitable clone is selected. A selected monoclonal cell line may be further subjected to safety testing and cell banking before it is used for human therapy.

Preferably the cell lines used in the present invention are capable of surviving for extended periods (several months and up to one year or more) when transplanted as encapsulated cells in vivo. The cell lines are preferably also capable of maintaining a secretion of bioactive neuropeptide at a level sufficient to ensure the therapeutic efficacy for a period greater than one month, preferably greater than three months, more preferably greater than six months. It is also preferable that the cells are capable of maintaining a relevant secretion of bioactive neuropeptide after encapsulation for at least one month, more preferably at least three months, more preferably at least six months.

The level of secretion preferably is at least 0.5 ng biologically active neuropeptide per 10⁵ cells per 24 hours is at least 0.5 ng, more preferably at least 0.75 ng, more preferably at least 1 ng, more preferably at least 2 ng, more preferably at least 2.5 ng, more preferably at least 5 ng, more preferably at least 7.5 ng, more preferably at least 10 ng, more preferably at least 15 ng, more preferably at least 20 ng, more preferably at least 25 ng, more preferably at least 50 ng.

When measured on a device level, the device (comprising encapsulated cells) is preferably capable of secreting in excess of 0.1 ng biologically active neuropeptide per 24 hours. More preferably, the amount of biologically active neuropeptide per 24 hours per device is at least 1 ng, more preferably at least 2 ng, more preferably at least 2.5 ng, more preferably at least 5 ng, more preferably at least 7.5 ng, more preferably at least 10 ng, more preferably at least 15 ng, more preferably at least 20 ng, more preferably at least 25 ng. These numbers refer to cylindrical devices of 5-7 mm length having a inner diameter of 500-700 μm and being loaded with 50000 cells.

### Expression Vectors

Construction of vectors for recombinant expression of neuropeptides for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue specific regulatory sequences). It will be appreciated by those skilled in the art that the design of the expression vector can depend on such factors as the choice of the host cell to be transformed, the level of expression of protein desired, etc. The expression vectors used in the invention can be introduced into host cells to thereby produce neuropeptide and neuropeptide mutants and variants encoded by nucleic acids as described herein.

In a preferred embodiment of the invention, the cells are transfected with a non-viral expression vector. The use of a non-viral expression vector is preferred for reasons of safety once the cells are implanted into a recipient subject.

In a preferred embodiment, the expression vector is a mammalian plasmid expression vector. Examples of mammalian plasmid expression vectors include pCDM8 (Seed, 1987. Nature 329: 840), pCl (Promega Inc), pSI (Promega), pNS (Example 1), pUbi1z (Johansen et al 2003, J Gene Medicine, 5:1080-1089), and pMT2PC (Kaufman, et al., 1987. EMBO J. 6: 187 195). When used in mammalian cells, the expression vector's control functions are often provided by viral regulatory elements. For other suitable expression systems for eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters and the UbiC promoter derived from human ubiquitin C (WO 98/32869).

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: CMV/chicken beta-actin (CAG) composite promoter, ubiquitin promoter, CMV promoter, JeT promoter (U.S. Pat. No. 6,555,674), SV40 promoter, Mt1 promoter, and Elongation Factor 1 alpha promoter (EF-1alpha). A particularly preferred promoter is one which is not subject to down regulation in vivo.

Examples of inducible/repressible promoters include: Tet-On, Tet-Off, Rapamycin-inducible promoter, Mx1.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Kozak consensus sequence, Woodchuck hepatitis virus post-transcriptional regulation element, WPRE, SP163 enhancer, CMV enhancer, non-translated 5′ or 3′ regions from the tau, TH or APP genes, and Chicken [beta]-globin insulator or other insulators. Preferable enhancing elements include Kozak consensus sequence, WPRE and beta-globin insulator.

### Transposon-Based Vectors

The “standard” types of plasmid and viral vectors that have previously been almost universally used for genetic transformation and transduction, have low efficiencies and may constitute a major reason for the low rates of transformation previously observed. The DNA (or RNA) constructs previously used often do not integrate into the host DNA, or integrate only at low frequencies. The present invention in one embodiment provides transposon-based vectors that can increase the integration rate.

Transposon-based vectors may produce integration frequencies an order of magnitude greater than has been achieved with normal plasmid vectors.

The transposon-based vectors of the present invention include a transposase gene operably linked to a first promoter, and a coding sequence for the heterologous polypeptide of the invention operably-linked to a second promoter, wherein the coding sequence for the heterologous polypeptide and its operably-linked promoter are flanked by transposase insertion (or substrate) sequences (Inverted Terminal Repeats) recognized by the transposase.

Transposases and Insertion Sequences

In a further embodiment of the present invention, the transposase found in the transposase-based vector is an altered target site transposase and the insertion sequences are those recognized by the altered transposase. However, the transposase located in the transposase-based vectors is not limited to an altered ATS transposase and can be derived from any transposase. Transposases known in the prior art include those found in AC7, Tn5SEQ1, Tn916, Tn951, Tn1721, Tn2410, Tn1681, Tn1, Tn2, Tn3, Tn4, Tn5, Tn6, Tn9, Tn10, Tn30, Tn101, Tn903, Tn501, Tn1000 (.gamma.delta.), Tn1681, Tn2901, AC transposons, Mp transposons, Spm transposons, En transposons, Dotted transposons, Mu transposons, Ds transposons, dSpm transposons and I transposons.

In some embodiments, the transposon-based vectors are optimized for expression in a particular host by changing the methylation patterns of the vector DNA. The transposon-based vectors may also be methylated to resemble eukaryotic DNA for expression in a eukaryotic host.

Transposases and insertion sequences from other analogous eukaryotic transposon-based vectors that can also be modified and used are, for example, the Drosophila P element derived vectors disclosed in U.S. Pat. No. 6,291,243; the Drosophila mariner element described in Sherman et al. (1998); or the Sleeping Beauty transposon. See also Hackett et al. (1999); D. Lampe et al., 1999. Proc. Natl. Acad. Sci. USA, 96:11428-11433; S. Fischer et al., 2001. Proc. Natl. Acad. Sci. USA, 98:6759-6764; L. Zagoraiou et al., 2001. Proc. Natl. Acad. Sci. USA, 98:11474-11478; and D. Berg et al. (Eds.), Mobile DNA, Amer. Soc. Microbiol. (Washington, D.C., 1989). Further examples of transposons include Frog Prince, Minos, S, Paris, Ban, Trx, Eagle, Froggy, and Jumpy (see e.g. Ivics et al, 2006, Curr Gene Therapy, 6:593-607).

In a preferred embodiment the transposase is Sleeping Beauty. Thus in one embodiment the expression construct of the invention and its promoter and any optional expression enhancing sequences are located between two terminal inverted repeats which are substrates for a transposase, preferably wherein said terminal inverted repeats are substrates for the Sleeping Beauty transposase.

The nucleic acid coding for the transposase under the control of an operatively linked promoter, may be located on the same vector as the expression construct or on another vector, and preferably said transposase is Sleeping Beauty.

Many transposases recognize different insertion sequences, and therefore, it is to be understood that a transposase-based vector will contain insertion sequences recognized by the particular transposase also found in the transposase-based vector.

Sleeping Beauty (SB) is a member of the Tc1/mariner-like family of transposon resurrected from the fish genome and exhibits high transpositional activity in a variety of vertebrate cultured cell lines, embryonic stem cells and in both somatic and germ line cells of the mouse in vivo. Sleeping Beauty has already proved to be a valuable tool for functional genomics in several vertebrate model organisms and shows promise for human gene therapeutic applications (Ivics, Z. and Izsvak, Z. (2006), Curr. Gene Ther., 6: 593-607).

The SB transposon is described in U.S. Pat. No. 7,148,203 and U.S. Pat. No. 6,489,458. Hyperactive variants of the SB transposon is described in WO 2009/003671, resulting in an improvement of the already valuable SB system as a method for introducing DNA into a cell.

More preferably the SB transposase is a hyperactive transposase as described in WO 2009/003671. Hyperactive transposases include Sleeping Beauty variants is selected from variants of SB10X comprising the amino acid sequence differing from SEQ ID NO 24 by 1 to 20 amino acids. In a preferred embodiment, the hyperactive SB is variant 28 from WO 2009/003671, having the amino acid sequence set forth herein as SEQ ID NO 26. In a most preferred embodiment, the hyperactive SB is variant 27 from WO 2009/003671, having the amino acid sequence set forth herein as SEQ ID NO 25.

### Virus Vectors

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV.

A special and preferred type of retroviruses includes the lentiviruses which can transduce a cell and integrate into its genome without cell division. A lentivirus particle can be produced from a lentiviral vector comprising a 5′ lentiviral LTR, a tRNA binding site, a packaging signal, a promoter operably linked to a polynucleotide signal encoding neuropeptide, an origin of second strand DNA synthesis and a 3′ lentiviral LTR.

Retroviral vectors are the vectors most commonly used in human clinical trials, since they may carry 7-8 kb of heterologous DNA and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the cell's genome.

Three classes of retroviral particles have been described; ecotropic, which can infect murine cells efficiently, and amphotropic, which can infect cells of many species. The third class includes xenotropic retrovirus, which can infect cells of another species than the species which produced the virus. Their ability to integrate only into the genome of dividing cells has made retroviruses attractive for marking cell lineages in developmental studies and for delivering therapeutic or suicide genes to cancers or tumours.

The retroviral vectors preferably are replication defective. This prevents further generation of infectious retroviral particles in the target tissue—instead the replication defective vector becomes a “captive” transgene stably incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the L signal, and expressing the gag/pol genes and the env gene from separate transcriptional units. In addition, in some 2. and 3. generation retroviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses, see, e.g. U.S. Pat. No. 4,861,719 herein incorporated by reference.

### Introns

In a preferred embodiment of the invention, the expression construct coding for neuropeptide or a neuropeptide variant includes an intron in the transcript. The highest producing cell lines have been obtained with intron-containing expression constructs.

From an analysis of the human genome at Gen Bank it can be derived that the smallest known intron is 4 bp and the longest known intron is 1,022,077 bp. Based on this knowledge, it is contemplated that the length of the intron used in the context of the present invention can be varied considerably. Except from the upper known limit given by Genbank, it is difficult to give any upper limit for the length of an intron, which is functional in the context of the present invention. In the broadest possible context there is no upper limit for the length of the intron, as long as it can be successfully cloned into the expression vector. For practical reasons one of skill in the art would select an intron, which is less than 100,000 bp long, more preferably less than 10,000 bp long.

The only parts of an intron that are really highly conserved are the sequences immediately within the intron. This identifies the formula of a generic intron as: GT . . . AG

The ends are named proceeding from left to right along the intron, that is as the left (or 5′) and right (or 3′) splicing sites. Sometimes they are referred to as the donor and acceptor sites. The bases immediately adjacent the donor and acceptor sites are less conserved. The frequency of different bases at specific positions relative to the splicing sites follows the following percentages (Lewin B, Genes V, Oxford University Press, Oxford, 1994, page 914):

| Exon | | Intron | | | | | | | | | Exon |
|---|---|---|---|---|---|---|---|---|---|---|---|
| A | G | G | T | A | A | G | T------C | | A | G | N |
| 64% | 73% | 100% | 100% | 62% | 68% | 84% | 63% | 65% | 100% | 100% | |

The sequence within these splicing sites can for every single intron be varied considerably by addition, deletion or substitution of bases. In a preferred embodiment of the invention the intron comprises a nucleotide sequence which is derived from a naturally occurring intron, and which has at least 50% sequence identity to said naturally occurring intron. More preferably, the intron has at least 60% sequence identity to said naturally occurring intron, more preferably at least 70%, more preferably at least 75%, more preferably at least 80%, more preferably at least 85%, more preferably at least 90%, more preferably at least 95%, more preferably at least 98%. The higher % sequence identities are preferred as less work is required to assemble to expression construct, and as the possibility of changing the function of the intron increases with the number of differences between a naturally occurring intron and a variant thereof.

In a preferred embodiment the intron is shorter such as less than 10,000 bp, which will considerably ease the cloning. Accordingly the intron may be less than 9,000 bp long, preferably less than 8,000, more preferably less than 7,000, more preferably less than 6,000, more preferably less than 5,000, more preferably less than 4,500, more preferably less than 4,000, more preferably less than 3,500, more preferably less than 3,000, more preferably less than 2,500, more preferably less than 2,000, more preferably less than 1,500, more preferably less than 1,000, such as less than 750, for example less than 500, such as less than 250, for example less than 200.

Similarly, it is expected that the intron should have a certain length to be properly spliced out from the transcript before translation. Therefore preferably the intron is more than 4 bp long, such ad more than 10 bp long, for example more than 20 bp long, preferably more than 50 bp long, more preferably more than 75 bp long.

An intron may be from 4 bp to 1 mio by long, more preferably from 10-10,000 bp, more preferably from 20-2000 bp, for example from 50-1500 bp, such as preferably from 75-200 bp, for example preferably from 500-1500 bp. The preferred introns of the present invention lie in the range from 100 to 1000 bp.

The origin of the intron may be any. It may also be a synthetic intron as long as it functions as an intron. As the present invention concerns human cell lines, it is preferable to use an intron from a species that is as closely related to human beings as possible. Therefore, preferably the intron is of eukaryotic origin. More preferably the intron is of mammalian origin. For example the intron may be of rodent origin or of primate origin. Still more preferably, the intron is of human origin.

It is preferred to have the intron located in the 5′ UTR or in the part of the coding sequence closest to the start codon, i.e. the first part of the coding sequence. Cloning is easier when the intron is placed in the 5′ UTR of the transcript. It is contemplated by the present inventors that a similar effect may be obtained by cloning an intron into the sequence coding for neuropeptide. In the case of cloning inside the coding sequence, the intron is preferably placed in the part of the coding sequence closest to the start codon, i.e. the first part of the coding sequence.

In a preferred embodiment the intron is derived from a first intron. A first intron is the intron located closest to the transcription start site in the gene from which it is derived. While first introns are preferred, it is to be understood that any intron such as a second, third, fourth, fifth, or sixth intron may also be used. A first intron of a particular gene may be referred to as intron A.

It is expected that it is sufficient to include one intron in the expression constructs in the human cell lines of the present invention. Including further introns is of course possible, and is also contemplated by the present inventors. In principle there is no upper limit to the number of introns inserted into the transcript but for practical reasons, the skilled practitioner would choose to keep the number as low as possible to keep the length of the expression construct within practical limits. The number of introns may be two, three, four, five or even higher.

One particularly preferred intron is the chimeric intron included in the pCl expression vector available from Promega Corp, Madison Wis., USA (Catalogue no.: E1731). This intron is composed of the 5′-donor site from the first intron of the human b-globin gene and the branch and 3′-acceptor site from the intron that is between the leader and the body of an immunoglobulin gene heavy chain variable region (Bothwell et al, 1981, Cell 24:625). The sequences of the donor and acceptor sites along with the branchpoint site have been changed to match the consensus sequences for splicing (Senaphthy et al, 1990, Meth. Enzymol. 183:252). The pCl expression vector is available from Promega Corp. The length of the intron is 113 bp. The sequence lying between the splice sites of the “pCl intron” can be varied. Preferably the intron comprises a sequence, which is derived from the “pCl intron”, which derived sequence has at least 50% sequence identity to the sequence set forth above. More preferably, the intron comprises a sequence having at least 60% sequence identity to said “pCl intron”, more preferably at least 70%, more preferably at least 75%, more preferably at least 80%, more preferably at least 85%, more preferably at least 90%, more preferably at least 95%, more preferably at least 98%.

Another preferred intron is derived from insulin. Preferably rodent insulin II, more preferably rat preproinsulin II intron A (bases no. 982.1100 of GenBank acc. # J00748). The sequence lying between the splice sites of rat insulin II intron A can be varied. Preferably the intron comprises a sequence, which is derived from rat insulin II A intron and therefore has at least 50% sequence identity to the sequence set forth above. More preferably, the intron comprises a sequence having at least 60% sequence identity to said sequence, more preferably at least 70%, more preferably at least 75%, more preferably at least 80%, more preferably at least 85%, more preferably at least 90%, more preferably at least 95%, more preferably at least 98%.

A further preferred intron is the ubiquitin promoter intron, preferably the human ubiquitin C promoter intron (Johansen et al. 1990, FEBS Lett. 267, 289-294). The UbiC intron is 811 bp long (bases no. 3959 . . . 4769 of GenBank acc # D63791). The ubiquitin C promoter intron is available from the pUbi1z expression vector described in Johansen et al 2003, J Gene Medicine, 5:1080-1089. The sequence lying between the splice sites of said ubiqutin intron can be varied. Preferably the intron comprises a sequence, which is derived from rat ubiquitin intron, and which has at least 50% sequence identity to the sequence set forth above. More preferably, the intron comprises a sequence which has at least 60% sequence identity to the sequence of said ubiquitin intron, more preferably at least 70%, more preferably at least 75%, more preferably at least 80%, more preferably at least 85%, more preferably at least 90%, more preferably at least 95%, more preferably at least 98%.

Another preferred intron is the EF-1alpha intron A (bases no. 609.1551 of Genbank accession number: J04617 J04616 Human elongation factor EF-1alpha gene, complete cds). This intron is 943 bp long. The sequence lying between the splice sites of said EF-1alpha intron A can be varied. Preferably the intron comprises a sequence which is derived from the EF-1alpha intron A, and which has at least 50% sequence identity to the sequence set forth above. More preferably, the intron comprises a sequence which has at least 60% sequence identity to said EF-1alpha intron A, more preferably at least 70%, more preferably at least 75%, more preferably at least 80%, more preferably at least 85%, more preferably at least 90%, more preferably at least 95%, more preferably at least 98%.

Another preferred intron is the IgSP intron (SEQ ID No 5 compared to SEQ ID NO 6). The sequence lying between the splice sites of said IgSP intron can be varied. Preferably the intron comprises a sequence which is derived from the IgSP intron, and which has at least 50% sequence identity to the sequence set forth above. More preferably, the intron comprises a sequence which has at least 60% sequence identity to said IgSP intron, more preferably at least 70%, more preferably at least 75%, more preferably at least 80%, more preferably at least 85%, more preferably at least 90%, more preferably at least 95%, more preferably at least 98%.

Preferably the intron is selected from the group consisting of the IgSP intron, pCl intron, the ratINS-intrA, and the Ubiquitin intron; and a sequence variant having at least 80% sequence identity to the sequence of any of said introns.

More preferably the intron is selected from the group consisting of the pCl intron and sequence variants thereof having at least 80% sequence identity to the sequence of said intron.

### Encapsulation

Encapsulated cell biodelivery is based on the concept of protecting cells from the recipient host's immune system by surrounding the cells with a semipermeable biocompatible material before implantation within the host. The invention includes a device in which cells are encapsulated in an immunoisolatory device. An “immunoisolatory device” means that the device, upon implantation into a recipient host, minimises the deleterious effects of the host's immune system on the cells in the core of the device. Cells are immunoisolated from the host by enclosing them within implantable polymeric devices formed by a microporous membrane. This approach prevents the cell-to-cell contact between host and implanted tissues, eliminating antigen recognition through direct presentation. The membranes used can also be tailored to control the diffusion of molecules, such as neuropeptide, based on their molecular weight. Using encapsulation techniques, cells can be transplanted into a host without immune rejection, either with or without use of immunosuppressive drugs. Useful biocompatible polymer devices usually contain a core that contains cells, either suspended in a liquid medium or immobilised within an immobilising matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the device, thereby protecting the encapsulated cells from immune destruction. The semipermeable nature of the device membrane also permits the biologically active molecule of interest to easily diffuse from the device into the surrounding host tissue.

The device can be made from a biocompatible material. A “biocompatible material” is a material that, after implantation in a host, does not elicit a detrimental host response sufficient to result in the rejection of the device or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably the device of this invention will be similar to those described by WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050. Such devices allow for the passage of metabolites, nutrients and therapeutic substances while minimizing the detrimental effects of the host immune system. Components of the biocompatible material may include a surrounding semipermeable membrane and an internal cell-supporting scaffolding. Preferably, the recombinant cells are seeded onto the scaffolding, which is encapsulated by the permselective membrane. The filamentous cell-supporting scaffold may be made from any biocompatible material selected from the group consisting of acrylic, polyester, polyethylene, polyvinylalcohol, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which seeded cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

The jacket preferably has a molecular weight cutoff, defined as that molecular weight, where the membrane (the jacket) will reject 90% of the solutes of less than 1000 kD, more preferably between 50-700 kD, most preferably between 70-300 kD. The molecular weight cutoff should be selected to ensure that the bioactive neuropeptide can escape from the device while protecting the encapsulated cells from the immune system of the patient.

The thickness of the jacket typically lies in the range of 2 to 200 microns, more preferably from 50 to 150 microns. The jacket should have a thickness to give the device sufficient strength to keep the cells encapsulated and should with this in mind be kept as thin as possible to take up as little space as possible.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane may be formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The device can be any configuration appropriate for maintaining biological activity and providing access for delivery of the product or function, including for example, cylindrical, rectangular, disk-shaped, patch-shaped, ovoid, stellate, or spherical. Moreover, the device can be coiled or wrapped into a mesh-like or nested structure. If the device is to be retrieved after it is implanted, configurations, which tend to lead to migration of the devices from the site of implantation, such as spherical devices small enough to travel in the recipient host's blood vessels, are not preferred. Certain shapes, such as rectangles, patches, disks, cylinders, and flat sheets offer greater structural integrity and are preferable where retrieval is desired. A particularly preferred shape is cylinder-shaped as such a shape is easily produced from hollow fibers which can be produced industrially.

When macrocapsules are used, preferably at least 10³ cells are encapsulated, such as between 10³ and 10⁸ cells are encapsulated, most preferably 10⁴ to 10⁷ cells are encapsulated in each device. Of course, the number of cells in each device depends on the size of the device. As a rule of thumb, in a device with foam (described below) the present inventors have found that loading between 10,000 and 250,000 cells per μL of device (volume calculated as the volume inside the outer jacket, i.e. including scaffolding/matrix), more preferably from 50,000 to 200,000 cells per μL, more preferably from 100,000 to 200,000 cells per μL. The number of cells to be loaded also depends on the size of the cells.

Dosage may be controlled by implanting a fewer or greater number of devices, preferably between 1 and 10 devices per patient.

A macrocapsule in the present context is a device having a volume of at least 0.5 μL, such as from 1 to 10 μL.

The length of the active part of the macrocapsule preferably is from 5 to 20 mm. The active part is the part of the macrocapsule comprising cells.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the devices may be used, including the use of polymer adhesives or crimping, knotting and heat sealing. In addition, any suitable “dry” sealing method can also be used, as described, e.g., in U.S. Pat. No. 5,653,687, incorporated by reference.

The encapsulated cell devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see WO 97/34586, incorporated by reference).

### Foam Scaffolds:

The foam scaffold may be formed from any suitable material that forms a biocompatible foam with an open cell or macroporous structure with a network of pores. An open-cell foam is a reticulate structure of interconnected pores. The foam scaffold provides a non-biodegradable, stable scaffold material that allows attachment of adherent cells. Among the polymers that are useful in forming the foam scaffolds for the devices of this invention are thermoplastics and thermoplastic elastomers.

Some examples of materials useful in forming suitable foam scaffolds are listed in Table 1.

TABLE 1

| Thermoplastic | |
|---|---|
| Thermoplastics: | Elastomers: |
| Acrylic | Polyamide |
| Modacrylic | Polyester |
| Polyamide | Polyethylene |
| Polycarbonate | Polypropylene |
| Polyester | Polystyrene |
| Polyethylene | Polyurethane |
| Polypropylene | Polyvinyl Alcohol |
| Polystyrene | Silicone |
| Polysulfone | |
| Polyethersulfone | |
| Polyvinylidene fluoride | |

Thermoplastic foam scaffolds made from polysulfone and polyethersulfone, and thermoplastic elastomer foam scaffolds made from polyurethane and polyvinyl alcohol are preferred.

The foam must have some (but not necessarily all) pores of a size that permits cells to attach to the walls or surfaces within the pores. The pore size, pore density and void volume of the foam scaffold may vary. The pore shape may be circular, elliptical or irregular. Because the pore shape can vary considerably, its dimensions may vary according to the axis being measured. For the purposes of this invention, at least some pores in the foam should have a pore diameter of between 20-500 μm, preferably between 50-150 μm. Preferably the foregoing dimensions represent the mean pore size of the foam. If non-circular, the pore may have variable dimensions, so long as its size is sufficient to permit adherent cells to attach to the walls or surfaces within the pore. In one embodiment, foams are contemplated having some elliptical pores that have a diameter of 20-500 μm along the minor axis and a diameter of up to 1500 μm along the major axis.

In addition to the foregoing cell permissive pores sizes, preferably a least a fraction of the pores in the foam should be less than 10 μm to be cell impermissive but still provide channels for transport of nutrients and biologically active molecules throughout the foam. Pore density of the foam (i.e., the number per volume of pores that can accommodate cells, as described above) can vary between 20-90%, preferably between 50-70%. Similarly, the void volume of the foam may vary between 20-90%, preferably between 30-70%.

The walls or surfaces of the pores may be coated with an extracellular matrix molecule or molecules, or other suitable molecule. This coating can be used to facilitate adherence of the cells to the walls of the pores, to hold cells in a particular phenotype and/or to induce cellular differentiation.

Preferred examples of extracellular matrix molecules (ECM) that can be adhered to the surfaces within the pores of the foams include: collagen, laminin, vitronectin, polyornithine and fibronectin. Other suitable ECM molecules include glycosaminoglycans and proteoglycans; such as chrondroitin sulfate, heparin sulfate, hyaluron, dermatan sulfate, keratin sulfate, heparan sulfate proteoglycan (HSPG) and elastin.

The ECM may be obtained by culturing cells known to deposit ECM, including cells of mesenchymal or astrocyte origin. Schwann cells can be induced to synthesize ECM when treated with ascorbate and cAMP. See, e.g., Baron-Van Evercooren et al., “Schwann Cell Differentiation in vitro: Extracellular Matrix Deposition and Interaction,” Dev. Neurosci., 8, pp. 182-96 (1986).

In addition, adhesion peptide fragments, e.g., RGD containing sequences (ArgGlyAsp), YIGSR-containing sequences (TyrIleGlySerArg), as well as IKVAV containing sequences (IleLysValAlaVal), have been found to be useful in promoting cellular attachment. Some RGD-containing molecules are commercially available—e.g., PepTite-2000™ (Telios).

The foam scaffolds of this invention may also be treated with other materials that enhance cellular distribution within the device. For example, the pores of the foam may be filled with a non-permissive hydrogel that inhibits cell proliferation or migration. Such modification can improve attachment of adherent cells to the foam scaffold. Suitable hydrogels include anionic hydrogels (e.g., alginate or carageenan) that may repel cells due to charge. Alternately, “solid” hydrogels (e.g., agarose or polyethylene oxide) may also be used to inhibit cell proliferation by discouraging binding of extracellular matrix molecules secreted by the cells.

Treatment of the foam scaffold with regions of a non-permissive material allows encapsulation of two or more distinct cell populations within the device without having one population overgrow the other. Thus non-permissive materials may be used within the foam scaffold to segregate separate populations of encapsulated cells. The distinct populations of cells may be the same or different cell types, and may produce the same or different biologically active molecules. In one embodiment, one cell population produces a substance that augments the growth and/or survival of the other cell population. In another embodiment, multiple cell types producing multiple biologically active molecules are encapsulated. This provides the recipient with a mixture or “cocktail” of therapeutic substances.

The devices of this invention may be formed according to any suitable method. In one embodiment, the foam scaffold may be pre-formed and inserted into a pre-fabricated jacket, e.g., a hollow fiber membrane, as a discrete component.

Any suitable thermoplastic or thermoplastic elastomer foam scaffold material may be preformed for insertion into a pre-fabricated jacket. In one embodiment we prefer polyvinyl alcohol (PVA) sponges for use as the foam scaffold. Several PVA sponges are commercially available. For example, PVA foam sponges #D-3, 60 μm pore size are suitable (Rippey Corp, Kanebo). Similarly, PVA sponges are commercially available from Ivalon Inc. (San Diego, Cailf.). PVA sponges are water-insoluble foams formed by the reaction of aerated Poly(vinyl alcohol) solution with formaldehyde vapor as the crosslinker. The hydroxyl groups on the PVA covalently crosslink with the aldehyde groups to form the polymer network. The foams are flexible and elastic when wetted and semi-rigid when dried.

As an alternative, support a mesh or yarn may be used as described in U.S. Pat. No. 6,627,422.

For easy retrival and for fastening the device to the skull, the device may be equipped with a tether anchor. Similarly, for easy retrieval and fastening to the eye, the device may be equipped with a suture eyelet.

For implantation into the CNS or the spinal cord, the tether is preferably equipped with a stiffener as described in WO 2006/122551.

Capsules may be filled as using a syringe as described in the examples. Alternatively, automated or semi-automated filling may be used.

### Microcapsules

In addition to the macrocapsules described above, the neuropeptide secreting cells of the present invention may be encapsulated in microcapsules or microspheres. Microcapsules or microspheres as defined herein are capsules holding less than 10⁴ cells per capsule. Microcapsules may contain substantially less than 10⁴ cells, such as less than 1000 cells per capsule for example less than 100 cells per capsule, such as less than 50 cells per capsule, for example less than 10 cells per capsule, such as less than 5 cells per capsule. Such microcapsules may be structurally relatively simple in that they contain cells dispersed more or less uniformly inside a matrix. Microcapsules may also be coated to provide a more two-layered structure and to ensure that no cells project through the surface of the microcapsules. As the microcapsules typically are small (diameter typically less than 250 μm, such as less than 150 μm, for example less than 100 μm, such as less than 50 μm, for example less than 25 μm) they can be handled like a liquid suspension and be injected at a treatment site.

### Support Matrix for Neuropeptide Producing Cells

The method of the present invention further comprises culturing of the neuropeptide producing cells in vitro on a support matrix prior to implantation into the mammalian brain. The preadhesion of cells to microcarriers prior to implantation in the brain is designed to enhance the long-term viability of the transplanted cells and provide long term functional benefit. Methods for culturing cells on a support matrix and methods for implanting said cells into the brain are described in U.S. Pat. No. 5,750,103 (incorporated by reference).

To increase the long term viability of the transplanted cells, the cells to be transplanted can be attached in vitro to a support matrix prior to transplantation. Materials of which the support matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances, or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of the cells. Thus, for example, the matrix material may be extracellular matrix or basement membrane material, which is produced and secreted by cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

When attached to the support according to the present invention, the cells used for transplantation are generally on the “outer surface” of the support. The support may be solid or porous. However, even in a porous support, the cells are in direct contact with the external milieu without an intervening membrane or other barrier. Thus, according to the present invention, the cells are considered to be on the “outer surface” of the support even though the surface to which they adhere may be in the form of internal folds or convolutions of the porous support material which are not at the exterior of the particle or bead itself.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, see, for example, Fisher Biotech Source 87-88, Fisher Scientific Co., 1987, pp. 72-75; Sigma Cell Culture Catalog, Sigma Chemical Co., St, Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### Suicide Systems

The devices of the present invention, which encapsulate neuropeptide-secreting cells, may be retrieved from the patient when required. As a further safety precaution the cells may be equipped with a suicide system, which ensures that the cells may be selectively killed upon administration of a suitable drug to the patient in question.

The suicide system is particularly preferred for naked cell transplantation according to the present invention, as the possibilities for removing naked cells after transplantation are very limited.

One such suicide system is based on thymidine kinases. By having a built-in suicide system in which a thymidine kinase is expressed constitutively or inducibly, the cells can be killed by administering to the individual a therapeutically effective amount of a nucleoside analog, such as AZT. The nucleoside analogue can be administered if the encapsulated cells start to proliferate in an uncontrolled manner. One may also wish to terminate the treatment simply because there is no need for the neuropeptide-secreting cells anymore, because termination must be immediate and cannot await surgical removal of the encapsulated cells or because further treatment is by some other route.

In the cases where transplanted or encapsulated cells have been conditionally immortalised before transplantation there is a theoretical risk that the oncogene initiates transcription after transplantation and that the transplanted cells consequently become tumorigenic. Whenever cells are immortalised by transduction with an oncogene under the control of an inducible promoter (e.g. the Tet on-off system, the Mx1 promoter or the like), a thymidine kinase (TK) enzyme coding sequence may be inserted into the vector construct under the control of the same promoter (e.g. by using an IRES construct) or the TK coding sequence may be inserted into another vector with an identical promoter. This ensures that whenever the oncogene is transcribed, the TK is also transcribed and the transduced and tumorigenic cells can be selectively killed by administering a prodrug.

There are several examples of thymidine kinase (TK) genes described in the art. One preferred TK is the HSV-thymidine kinase. Other preferred kinases include Drosophila melanogaster thymidine kinase described in Munch-Petersen et al 2000, J. Biol. Chem. 275:6673-6679. Mutants of this particular kinase are even more preferred as they have decreased LD₅₀ with respect to several nucleoside analogues (WO 01/88106). Another group of preferred thymidine kinases include plant kinases described in WO 03/100045.

### Immunostimulatory Cell Surface Proteins

In one embodiment there is provided encapsulated human cells capable of expressing an immunostimulatory cell surface polypeptide in addition to neuropeptide or a neuropeptide variant. These immunostimulatory cell surface expressing cells are particularly useful when encapsulated for implantation in a human patient, because cells escaping from a ruptured device are destroyed by the patient's immune system. A host immune response will not be triggered by the recombinant cells expressing an immunostimulatory cell surface polypeptide in an intact device. In case of a device failure, however, the released cells are effectively eliminated by phagocytes without complement activation or the creation of an immune memory.

In a specific embodiment, a chimeric polypeptide containing the human transferrin receptor membrane domain anchors a human IgG1 Fc to the surface of the cell plasma membrane in a “reversed orientation”, thus mimicking the configuration of IgG during opsonisation. The human IgG1 chimeric polypeptide binds the Fc receptor to activate phagocytes, such as macrophages, but avoids the undesirable characteristics of also activating the complement cascade (“complement fixation”). A chronically activated complement system can kill host cells, and accumulating evidence suggests that this mechanism can cause many degenerative diseases, including inflammation and neurodegenerative diseases. Further details of this embodiment of the invention are described in U.S. Pat. No. 6,197,294.

According to this embodiment the cell line further comprises a construct comprising a promoter operatively linked to a polynucleotide sequence encoding a fusion protein comprising an immunostimulatory cell surface protein linked at the amino terminus to a second cell surface polypeptide, wherein the second cell surface polypeptide comprises a transmembrane region, wherein upon expression, the fusion protein is expressed on the cell surface.

Preferably the immunostimulatory cell surface polypeptide activates phagocytes but does not fix complement. In one embodiment the immunostimulatory cell surface polypeptide is a region of IgG, preferably Fc. The second cell surface polypeptide may be a transferrin receptor hinge region.

### Neurological Disorders

Neuropeptides in general can be used to treat, alleviate or prevent one or more symptoms of a disease of the central or peripheral nervous system by localised delivery.

The present invention contemplates delivery of the neuropeptide-encoding vectors, neuropeptide-secreting cells or devices secreting neuropeptide by injection or implantation of a composition of a gene therapy vector, of naked cells genetically modified by the vectors of the present invention or by implantation of devices with cells modified to secrete neuropeptides of the invention.

According to this invention, capsular delivery of neuropeptide, synthesised by human cells in vivo, to the brain ventricles, brain parenchyma, or other suitable CNS location, ranging from 1-1500 ng/day is contemplated. The actual dosage of neuropeptide can be varied by implanting high or low producing clones, more or less cells or fewer or greater number of devices. We contemplate delivery of 0.1-1500, preferably 1 to 1000, more preferably 10-600, most preferably 50-500, ng neuropeptide/human/day, for ventricular delivery and 0.1-1500, preferably 10-150 ng neuropeptide/human/day for parenchymal delivery.

Intraocularly, preferably in the vitreous, we contemplate delivery of 50 pg to 500 ng, preferably from 100 pg to 100 ng, and most preferably from 1 ng to 50 ng per eye per patient per day. For periocular delivery, preferably in the sub-Tenon's space or region, slightly higher dosage ranges are contemplated of up to 1 μg per eye per patient per day.

In one embodiment, genetically modified human cells secreting human neuropeptide are encapsulated in semipermeable membranes, and implanted intraocularly, intraventricularly or intraparenchymally in a suitable mammalian host, preferably a primate, most preferably a human.

Accordingly, neuropeptide-expressing cell lines and neuropeptide-encoding vectors of the invention are believed to be useful in promoting the development, maintenance, or regeneration of neurons in vivo, including central (brain and spinal chord), peripheral (sympathetic, parasympathetic, sensory, and enteric neurons), and motorneurons. Neuropeptide-expressing cell lines and neuropeptide-encoding vectors of the invention are utilised in methods for the treatment of a variety of neurologic diseases and disorders. In a preferred embodiment, the cell lines and vectors of the present invention are administered to a patient to treat neurological disorders. By “neurological disorders” herein is meant disorders of the central and/or peripheral nervous system that are associated with neuron degeneration or damage or loss of neurons.

### Medical Use of Galanin Expressing Vectors and Galanin Secreting Cells

Galanin is know to have therapeutic potential in the treatment of including seizure, Alzheimer's disease, mood disorders, anxiety, alcohol intake in addiction, metabolic diseases, pain and solid tumors (Mitsukawa et al 2008, Cell Mol Life Sci, 65:1796-1805). In particular, Galanin is known to be useful for treating Epilepsy (Lerner et al, Cell Mol Life Sci, 2008, 65:1864-1871).

Therefore Galanin-expressing cell lines, and galanin encoding gene therapy vectors of the invention can be used to treat human neurodegenerative disorders, such as Alzheimer's disease and other dementias, epilepsy, Huntington's disease, and other conditions characterized by necrosis or loss of neurons or their processes, whether in the brain, brain stem, spinal cord and/or the peripheral nerves.

In another embodiment Galanin-expressing cell lines, and galanin encoding gene therapy vectors can be used in the treatment of neuropsychiatric disorders including but not limited to depression, such as medically intractable depression, obsessive compulsory disorder (OCD), Tourette's syndrome, anxiety, bipolar disorders, and phobia.

In one embodiment, because galanin is neuroprotective, the cell lines and vectors can be used to treat damaged nerves due to trauma, burns, kidney disfunction, injury, and the toxic effects of chemotherapeutics used to treat cancer or AIDS.

Galanin-expressing cell lines and galanin-encoding vectors of the invention are particularly useful for treating Epilepsy, Huntington's Disease and Alzheimer's Disease.

For example, peripheral neuropathies associated with certain conditions, such as neuropathies associated with diabetes, AIDS, or chemotherapy may be treated using the formulations of the present invention.

Further, galanin-secreting cell lines or devices and galanin-expressing cell lines of the invention implanted either in the peripheral tissues or within the CNS, are preferably used to treat neuropathy, and especially peripheral neuropathy and associated neuropathic pain. “Peripheral neuropathy” refers to a disorder affecting the peripheral nervous system, most often manifested as one or a combination of motor, sensory, sensorimotor, or autonomic neural dysfunction. The wide variety of morphologies exhibited by peripheral neuropathies can each be attributed uniquely to an equally wide number of causes. For example, peripheral neuropathies can be genetically acquired, can result from a systemic disease, or can be induced by a toxic agent. Examples include, but are not limited to, diabetic peripheral neuropathy, distal sensorimotor neuropathy, AIDS-associated neuropathy, or autonomic neuropathies such as reduced motility of the gastrointestinal tract or atony of the urinary bladder. Examples of neuropathies associated with systemic disease include post-polio syndrome; examples of hereditary neuropathies include Charcot-Marie-Tooth disease, Refsum's disease, Abetalipoproteinemia, Tangier disease, Krabbe's disease, Metachromatic leukodystrophy, Fabry's disease, and Dejerine-Sottas syndrome; and examples of neuropathies caused by a toxic agent include those caused by treatment with a chemotherapeutic agent such as taxol, vincristine, cisplatin, methotrexate, or 3′-azido-3′-deoxythymidine.

A therapeutically effective dose of galanin-secreting cells or devices or galanin-encoding vector is administered to a patient. By “therapeutically effective dose” herein is meant a dose that produces the effects for which it is administered or that amount which provides therapeutic effect in a particular administration regimen. Dosage of galanin released from the cell lines or devices or vectors of the present invention is that needed to achieve an effective concentration of galanin in vivo, for the particular condition treated, though the dosage varies with the type of galanin variant, the desired duration of the release, the target disease, the subject animal species and other factors, such as patient condition. The exact dose will depend on the disorder to be treated and the implantation site, and will be ascertainable by one skilled in the art using known techniques.

In addition, as is known in the art, adjustments for age as well as the body weight, general health, sex, diet, time of administration, drug interaction and the severity of the disease may be necessary, and will be ascertainable with routine experimentation by those skilled in the art. Typically, the clinician will administer galanin-secreting cell lines or devices or galanin encoding vector of the invention until a dosage is reached that ameliorates, repairs, maintains, and/or, optimally, re-establishes neuron function. The dosage may also be a prophylactic dose which prevents or reduces degeneration of neurons. The progress of this therapy is easily monitored by conventional assays.

In the treatment of epilepsy the devices, cells or gene therapy vectors of the present invention are preferably implanted/injected in proximity of the seizure focus as measured by EEG, functional imaging or by electrodes. This may involve implanting/injecting into temporal lobe or the hippocampus.

In the treatment of Alzheimer's disease, it is contemplated to implant devices or cells or inject gene therapy vector into the cholinergic basal forebrain and/or possibly in the hippocampus.

In the treatment of neuropsychiatric disorders administration is preferably to the nucleus acumbens, the thalamus, and/or the frontal cortical regions.

In the treatment of Huntington's disease, we contemplate administration of devices/cells/gene therapy vector to the striatum and/or intracerebroventricularly.

In the treatment of pain, we contemplate administration to the spinal cord, the intrathecum and/or thalamus.

What has been stated about administration of galanin expressing vectors/cells is also valid for vectors/cells/devices of the invention encoding different neuropeptides intended for the same indications as galanin. The disorder is the primary factor deciding the site of administration.

### Ophthalmic Disorders

The neuropeptide releasing devices and cell lines of the present invention may be used to treat ophthalmic disorders such as described in U.S. Pat. No. 6,436,427 (incorporated by reference). Galanin has neuroprotective effects and may therefore be used in the treatment of disorders of the eye involving neuronal damage.

In general, devices are implanted into the vitreus humor of the eye to obtain administration to the retina. Devices are preferably inserted into the pars planum of the vitreous humor.

Retinopathy, e.g. diabetic retinopathy, is characterized by angiogenesis and retinal degeneration. Retinopathy includes, but is not limited to, diabetic retinopathy, proliferative vitreoretinopathy, and toxic retinopathy. Retinopathies may be treated by implanting devices intraocularly, preferably in the vitreous. We most prefer delivery into the vitreous for this indication. It may also be desirable to co-deliver one or more anti-angiogenic factors intraocularly, preferably intravitreally.

Uveitis involves inflammation and secondary degeneration that may affect retinal cells. This invention contemplates treating retinal degeneration caused by uveitis, preferably by vitreal or anterior chamber implantation of devices.

Retinitis pigmentosa, by comparison, is characterized by primary retinal degeneration. This invention contemplates treating retinitis pigmentosa by intraocular, preferably vitreal, implantation of devices.

Age-related macular degeneration involves both angiogenesis and retinal degeneration. Age-related macular degeneration includes, but is not limited to, dry age-related macular degeneration, exudative age-related macular degeneration, and myopic degeneration. This invention contemplates treating this disorder by implanting one or more devices intraocularly, preferably to the vitreous, and/or one or more anti-angiogenic factors intraocularly or periocularly.

Glaucoma is characterized by increased ocular pressure and loss of retinal ganglion cells. Treatments for glaucoma contemplated in this invention include delivery of neuropeptide that protect retinal cells from glaucoma associated damage, through intraocular, preferably intravitreal implantation.

Ocular neovascularization is a condition associated with many ocular diseases and disorders and accounting for a majority of severe visual loss. For example, we contemplate treatment of retinal ischemia-associated ocular neovascularization, a major cause of blindness in diabetes and many other diseases; corneal neovascularization, which predisposes patients to corneal graft failure; and neovascularization associated with diabetic retinopathy, central retinal vein occlusion, and possibly age-related macular degeneration.

In one embodiment of the present invention, cells of the invention are encapsulated and surgically inserted (under retrobulbar anesthesia) into the vitreous of the eye. For vitreal placement, the device may be implanted through the sclera, with a portion of the device or tether protruding through the sclera. Most preferably, the entire body of the device is implanted in the vitreous, with no portion of the device protruding into or through the sclera. Preferably the device is tethered to the sclera (or other suitable ocular structure). The tether may comprise a suture eyelet, or any other suitable anchoring means (see e.g. U.S. Pat. No. 6,436,427). The device can remain in the vitreous as long as necessary to achieve the desired prophylaxis or therapy. Such therapies for example include promotion of neuron or photoreceptor survival or repair, or inhibition and/or reversal of retinal neovascularization, as well as inhibition of uveal, retinal, and optic nerve inflammation.

### Gene Therapy

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells, which are then infused, grafted or otherwise transplanted into the patient, see, e.g. U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV.

Preferred viruses for treatment of disorders of the central nervous system are lentiviruses and adeno-associated viruses. Both types of viruses can integrate into the genome without cell divisions, and both types have been tested in pre-clinical animal studies for indications in the nervous system, in particular in the central nervous system.

Methods for preparation of AAV are described in the art, e.g. U.S. Pat. No. 5,677,158, U.S. Pat. No. 6,309,634, and U.S. Pat. No. 6,451,306 describe examples of delivery of AAV to the central nervous system.

A special and preferred type of retroviruses includes the lentiviruses which can transduce a cell and integrate into its genome without cell division. Thus preferably the vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors) and US 20020187951 (Lentiviral-mediated growth factor gene therapy for neurodegenerative diseases).

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome.

Three classes of retroviral particles have been described; ecotropic, which can infect murine cells efficiently, and amphotropic, which can infect cells of many species. A third class include xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors. These vectors may be particularly useful in the central nervous system for cancer treatment, where there is a relative lack of cell division in adult patients.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the ω signal, and expressing the gag/pol genes and the env gene from separate transcriptional units. In addition, in some 2. and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses. See, e.g., U.S. Pat. No. 4,861,719.

Numerous studies describe transduction of CNS cells using AAV or lentivirus expressing e.g. GDNF (Kordower, Ann Neurol, 2003 53 (suppl 3), s120-s134; WO 03/018821, Ozawa et al; US 2002187951, Aebischer et al; Georgievska et al 2002, Exp Nerol 117(2), 461-474; Georgievska et al 2002, NeuroReport 13(1), 75-82; Wang et al, 2002, Gene Therapy, 9(6), 381-389; US 2002031493, Rohne-Poulenc Rorer SA; U.S. Pat. No. 6,180,613 Roeckefeller University; Kozlowski et al 2000, Exp Neurol, 166(1), 1,15; Bensadoun 2000, Exp Neurol, 164(1), 15-24; Connor et al 1999, Gene Therapy, 6(12), 1936-1951; Mandel et al 1997, PNAS, 94(25), 14083-88; Lapchak et al 1997, Brain Research, 777 (1,2), 153-160; Bilang-Bleuel et al 1997, PNAS 94(16), 8818-8823). These and similar methods can be used in delivering neuropeptides to the central nervous system using the expression vectors of the present invention.

One important parameter for in vivo gene therapy is the selection of a suitable target tissue. A region of the brain is selected for its retained responsiveness to neuropeptides.

Importantly, specific in vivo gene delivery sites are selected so as to cluster in an area of neuronal loss. Such areas may be identified clinically using a number of known techniques, including magnetic resonance imaging (MRI) and biopsy. In humans, non-invasive, in vivo imaging methods such as MRI will be preferred. Once areas of neuronal loss are identified, delivery sites are selected for stereotaxic distribution so each unit dosage of gene therapy vector is delivered into the brain at, or within 500 μm from, a targeted cell, and no more than about 10 mm from another delivery site.

A further important parameter is the dosage of neuropeptide to be delivered into the target tissue. In this regard, “unit dosage” refers the number of viral particles/ml of gene therapy composition. Optimally, for delivery of neuropeptide using a viral expression vector, each unit dosage will comprise 2.5 to 25 μL of a gene therapy composition, wherein the composition includes a viral expression vector in a pharmaceutically acceptable fluid and provides from 10¹⁰ up to 10¹⁵ neuropeptide expressing viral particles per ml of gene therapy composition. Such high titers are particularly useful for adeno-associated virus. For lentivirus, the titer is normally lower, such as from 10⁸ to 10¹⁰ transducing units per ml (TU/mL).

In one embodiment, the administration site is the striatum of the brain, in particular the caudate and/or the putamen. Injection into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra. Injection may also be into both the striatum and the substantia nigra.

Within a given target site, the vector system may transduce a target cell. The target cell may be a cell found in nervous tissue, such as a neuron, astrocyte, oligodendrocyte, microglia or ependymal cell. In a preferred embodiment, the target cells are neurons.

The vector system is preferably administered by direct injection. Methods for injection into the brain (in particular the striatum) are well known in the art (Bilang-Bleuel et al (1997) Proc. Acad. Natl. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Stereotaxic injections are preferably given.

As mentioned above, for transduction in tissues such as the brain, it is preferable to use very small volumes, so the viral preparation is concentrated by ultracentrifugation. The resulting preparation may have at least 10⁸ t.u./ml, preferably from 10⁸ to 10¹⁰ t.u./ml, more preferably at least 10⁹ t.u./ml. (The titer is expressed in transducing units per ml (t.u./ml)). Improved dispersion of transgene expression may be obtained by increasing the number of injection sites and decreasing the rate of injection (Horellou and Mallet (1997) as above). Between 1 and 10 injection sites may be used, more commonly between 2 and 6. For a dose comprising 1−5×10⁹ t.u./ml, the rate of injection is preferably between 0.1 and 10 μl/min, usually about 1 μl/min.

Due to the high secretion efficiency of the improved vectors provided by the present invention, smaller volumes of virus composition need to be injected to obtain a clinical effect than if vectors comprising wild-type neuropeptide cDNA are used.

The gene therapy composition is delivered to each delivery cell site in the target tissue by microinjection, infusion, scrape loading, electroporation or other means suitable to directly deliver the composition directly into the delivery site tissue through a surgical incision. The delivery is accomplished slowly, such as over a period of about 5-10 minutes (depending on the total volume of gene therapy composition to be delivered).

Those of skill in the art will appreciate that the direct delivery method employed by the invention obviates a limiting risk factor associated with in vivo gene therapy; to wit, the potential for transduction of non-targeted cells with the vector carrying the neuropeptide encoding transgene. In the invention, delivery is direct and the delivery sites are chosen so diffusion of secreted neuropeptide takes place over a controlled and pre-determined region of the brain to optimise contact with targeted neurons, while minimizing contact with non-targeted cells.

### EXAMPLES

### Example 1

### Construction of Galanin Expression Plasmids for Production of Wt Galanin by Mammalian Cells

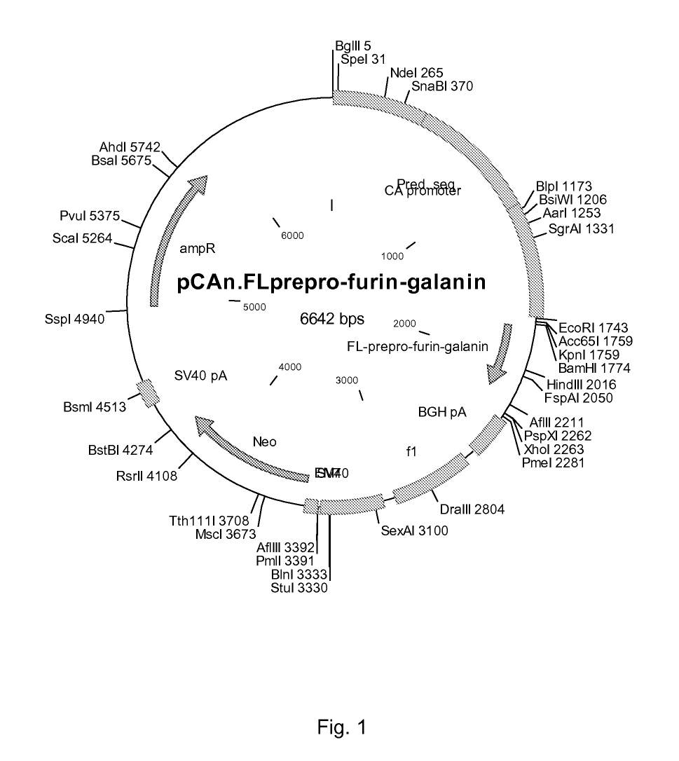

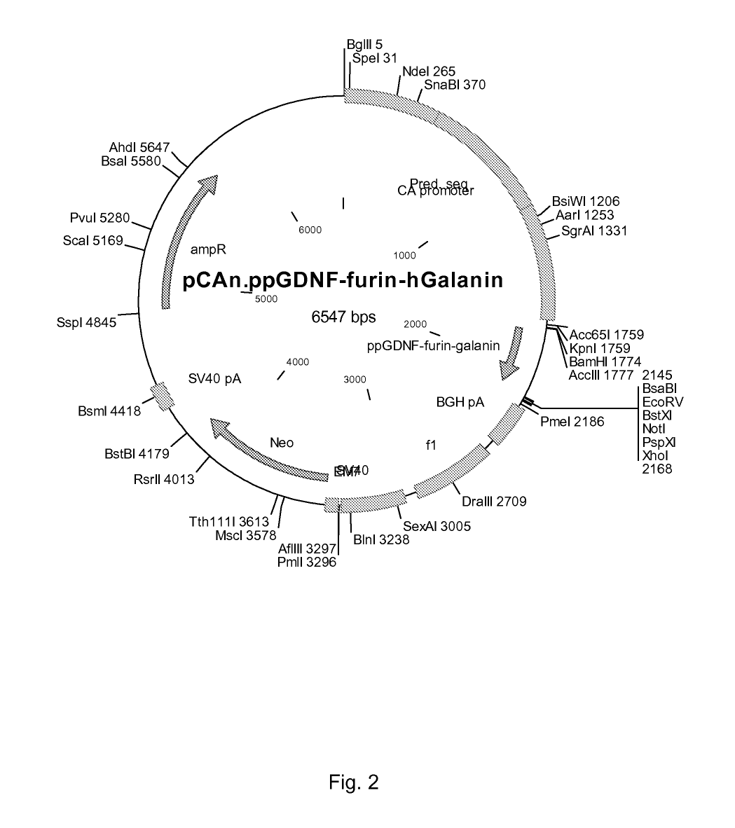

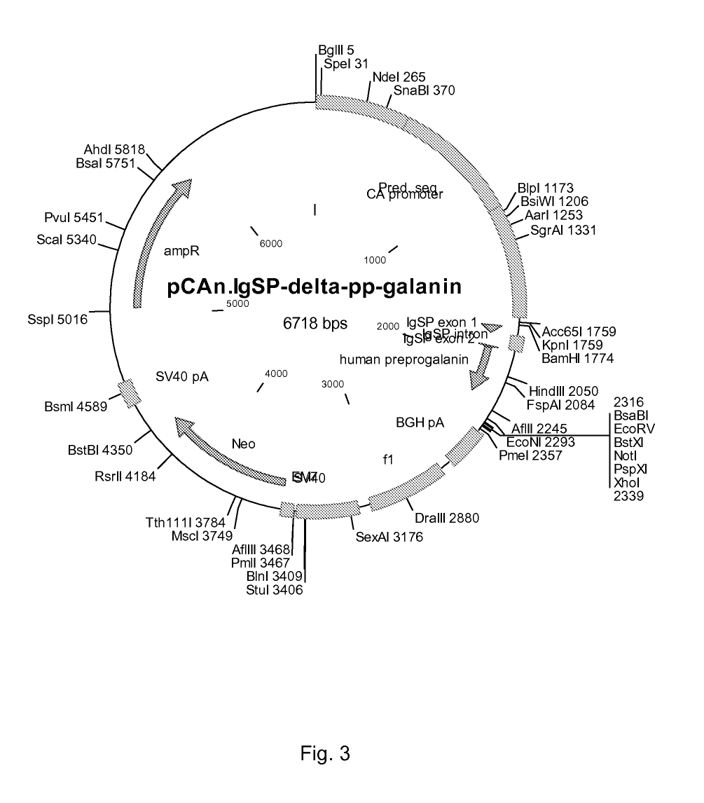

Three galanin constructs were made as follows:
1) FLprepro-furin-galanin was generated by overlapping PCR. In the first amplification step the prepro sequence of galanin—with the pro-convertase recognition sequence mutated into an optimal furin pro-convertase recognition consensus sequence—was PCR amplified from pCMV-SPORT6-hgalanin (obtained from RZPD Berlin, Germany, clone ID: IRATp970F0849D6) using the primers BamHI-preprogalanin, 5′ (SEQ ID NO 57) (5′-TATAGGATCCCCGCAGCTCAAGATG-3′) and Galanin prepro furin FLAP as (SEQ ID NO 58) (5′-CGTTTTTTTCTTGACGGCGACCAGAGCCCC-3′). In a second PCR reaction the furin FLAP-galanin fragment was amplified from pCMV-SPORT6-hgalanin using primers Furin FLAP-mature galanin s (SEQ ID NO 59) (5′-GTCGCCGTCAAGAAAAAAACGAGGCTGGACC-3′) and Preprogalanin-XhoI 3′ (SEQ ID NO 60) (5′-TATACTCGAGCAGGAATGGCTGACTC-3′). In the third step the products of step 1 and 2 were combined in a final PCR reaction that generated the FLprepro-furin-galanin fragment by using equimolar amounts of products of the first two PCR reactions and the primers BamHI-preprogalanin 5′ and Preprogalanin-XhoI 3′.
2) The pCAn vector is derived from pcDNA3.1 (Invitrogen). The CMV promoter was removed from pcDNA3.1 and replaced with the human CMV enhancer/chicken beta-actin (CAG) promoter and first intron. To generate a plasmid-based expression vector the resulting PCR fragment was cloned into pCAn digested with BamHI/XhoI. In this vector, the FLprepro-furin-galanin sequence is placed under transcriptional control of the CA promoter (chicken β-actin promoter with cytomegalovirus, CMV, enhancer) (see FIG. 1). Furthermore, the vector contains the Neo gene that confers G418 resistance when expressed in mammalian cells.
3) ppGDNF-furin-hGalanin was generated by overlapping PCR. In the first amplification step the prepro sequence of preproGDNF sequence—with a sub-optimal furin convertase recognition sequence mutated into an optimal furin recognition consensus sequence and a 10 bp galanin FLAP sequence in the 3′ end—was PCR amplified from pCln.preproGDNF-galanin (a plasmid generated in house from wt ppGDNF and galanin sequences) using the primers Bam-5′ preproGDNF s (SEQ ID NO 61) (5′-TATAGGATCCGGACGGGACTTTAAGATGAAG-3′) and 3′ppGDNF-furin-gaIFLAP as (SEQ ID NO 62) (5′-CCTTTTTTTTCTTGAACGGGTGGCTTGAATAAAATC-3′). In a second PCR reaction a fragment containing mature galanin with 10 bp preproGDNF-furin FLAP was amplified from pCln.preproGDNF-galanin using primers 5′ gal-furin-ppGDNF FLAP s (SEQ ID NO 63) (5′-CCCGTTCAAGAAAAAAAAGGGGCTGGACCC-3′) and Mature gala-STOP-XhoI 3′ (SEQ ID NO 64) (5′-TATACTCGAGTCAGCTGGTGAGGCCATTCTTGTCGC-3′). In the third step the products of step 1 and 2 were combined in a final PCR reaction that generated the ppGDNF-furin-hGalanin fragment by using equimolar amounts of products of the first two PCR reactions and the primers Bam-5′ preproGDNF s and Mature gala-STOP-XhoI 3′.
   To generate a plasmid-based expression vector the resulting PCR fragment was cloned into pCAn digested with BamHI/XhoI. In this vector, the ppGDNF-furin-hGalanin sequence is placed under transcriptional control of the CA promoter (see FIG. 2). Furthermore, the vector contains the Neo gene that confers G418 resistance when expressed in mammalian cells.
4) IgSP-deltaprepro-galanin was generated by overlapping PCR. In the first amplification step the galanin sequence coding for mature galanin and the C-terminal peptide with 10 bp IgSP FLAP (IgSP=mouse Ig heavy chain gene V-region signal peptide sequence) was PCR amplified from the pCMV-SPORT6-hgalanin plasmid using primers FLAP-IgSP-mature gala, 5′ (SEQ ID NO 65) (5′-GGTGAATTCGGGCTGGACCCTGAACAGCGCG-3′) and Deltaprepro-galanin-XhoI 3′ (SEQ ID NO 66) (5′-TATACTCGAGCAGGAATGGCTGACTCTGCATAAATTGGCC-3′). In a second PCR reaction a fragment containing the full length IgSP sequence with a 10 bp galanin FLAP from the 5′ end of mature galanin was amplified from pNUT-IgSP-hCNTF (U.S. Pat. No. 6,361,771) using primers IgSPkozak1s+BamHI (SEQ ID NO 67) (5′-TATAGGATCCGCCACCATGAAATGCAGCTGGGTTATC-3′) and IgSP-galanin FLAP as (SEQ ID NO 68) (5′-GGGTCCAGCCCGAATTCACCCCTGTAGAAAG-3′). In the third step the products of step 1 and 2 were combined in a final PCR reaction that generated the IgSP-deltaprepro-galanin fragment by using equimolar amounts of products of the first two PCR reactions and the primers IgSPkozak1s+BamHI and Deltaprepro-galanin-XhoI 3′.
   To generate a plasmid-based expression vector the resulting PCR fragment was cloned into pCAn digested with BamHI/XhoI. In this vector, the IgSP-deltaprepro-galanin sequence is placed under transcriptional control of the CA promoter (see FIG. 3). Furthermore, the vector contains the Neo gene that confers G418 resistance when expressed in mammalian cells.
      Sequences from the constructs are shown in Example 2.

### Example 2

### Sequences of the Chimeric Galanin Constructs

FLprepro-furin-qalanin nucleotide sequence present in
construct (SEQ ID NO 1)
ATGGCCCGAGGCAGCGCCCTCCTTCTCGCCTCCCTCCTCCTCGCCGCGGCCCTTTCTGCC
TCTGCGGGGCTCTGGTCGCCGTCAAGAAAAAAACGAGGCTGGACCCTGAACAGCGCGGGC
TACCTGCTGGGCCCACATGCCGTTGGCAACCACAGGTCATTCAGCGACAAGAATGGCCTC
ACCAGCAAGCGGGAGCTGCGGCCCGAAGATGACATGAAACCAGGAAGCTTTGACAGGTCC
ATACCTGAAAACAATATCATGCGCACAATCATTGAGTTTCTGTCTTTCTTGCATCTCAAA
GAGGCCGGTGCCCTCGACCGCCTCCTGGATCTCCCCGCCGCAGCCTCCTCAGAAGACATC
GAGCGGTCCT GA
FLprepro-furin-galanin is the full length wild type human
galanin including the pre- and pro-regions with the pro-
convertase cleavage site mutated into an optimal furin cleavage
consensus sequence.
Translation of FLprepro-furin-qalanin transcript (SEQ ID NO 2)
MARGSALLLASLLLAAALSASAGLWSPSRKKRGWTLNSAGYLLGPHAVGNHRSFSDKNGL
TSKRELRPEDDMKPGSFDRSIPENNIMRTIIEFLSFLHLKEAGALDRLLDLPAAASSEDI
ERS
The mature galanin sequence is accentuated in bold.
ppGDNF-furin-galanin nucleotide sequence present in construct
(SEQ ID NO 3)
ATGAAGTTATGGGATGTCGTGGCTGTCTGCCTGGTGCTGCTCCACACCGCGTCCGCCTTC
CCGCTGCCCGCCGGTAAGAGGCCTCCCGAGGCGCCCGCCGAAGACCGCTCCCTCGGCCGC
CGCCGCGCGCCCTTCGCGCTGAGCAGTGACTCAAATATGCCAGAGGATTATCCTGATCAG
TTCGATGATGTCATGGATTTTATTCAAGCCACCCGTTCAAGAAAAAAAAGGGGCTGGACC
CTGAACAGCGCGGGCTACCTGCTGGGCCCACATGCCGTTGGCAACCACAGGTCATTCAGC
GACAAGAATGGCCTCACCAGCTGA
ppGDNF-furin-galanin is the mature wild type human galanin
sequence fused to the prepro sequence of GDNF with the furin
recognition sequence mutated into an optimal furin consensus
sequence.
Translation of ppGDNF-furin-qalanin transcript (SEQ ID NO 4)
MKLWDVVAVCLVLLHTASAFPLPAGKRPPEAPAEDRSLGRRRAPFALSSDSNMPEDYPDQ
FDDVMDFIQATRSRKKRGWTLNSAGYLLGPHAVGNHRSFSDKNGLTS
The mature galanin sequence is accentuated in bold.
IgSP-galanin nucleotide sequence present in construct
(SEQ ID NO 5)
ATGAAATGCAGCTGGGTTATCTTCTTCCTGATGGCAGTGGTTACAGGTAAGGGGCTCCCA
AGTCCCAAACTTGAGGGTCCATAAACTCTGTGACAGTGGCAATCACTTTGCCTTTCTTTC
TACAGGGGTGAATTCGGGCTGGACCCTGAACAGCGCGGGCTACCTGCTGGGCCCACATGC
CGTTGGCAACCACAGGTCATTCAGCGACAAGAATGGCCTCACCAGCAAGCGGGAGCTGCG
GCCCGAAGATGACATGAAACCAGGAAGCTTTGACAGGTCCATACCTGAAAACAATATCAT
GCGCACAATCATTGAGTTTCTGTCTTTCTTGCATCTCAAAGAGGCCGGTGCCCTCGACCG
CCTCCTGGATCTCCCCGCCGCAGCCTCCTCAGAAGACATCGAGCGGTCCTGA
The IgSP-galanin contains an intron
Nucleotide sequence of spliced IgSP-galanin transcript
(SEQ ID NO 6):
ATGAAATGCAGCTGGGTTATCTTCTTCCTGATGGCAGTGGTTACAGGGGTCAATTCAGGC
TGGACCCTGAACAGCGCGGGCTACCTGCTGGGCCCACATGCCGTTGGCAACCACAGGTCA
TTCAGCGACAAGAATGGCCTCACCAGCAAGCGGGAGCTGCGGCCCGAAGATGACATGAAA
CCAGGAAGCTTTGACAGGTCCATACCTGAAAACAATATCATGCGCACAATCATTGAGTTT
CTGTCTTTCTTGCATCTCAAAGAGGCCGGTGCCCTCGACCGCCTCCTGGATCTCCCCGCC
GCAGCCTCCTCAGAAGACATCGAGCGGTCCTGA
Translation of spliced IgSP-galanin transcript (SEQ ID NO 7):
MKCSWVIFFLMAVVTGVNSGWTLNSAGYLLGPHAVGNHRSFSDKNGLTSKRELRPEDDMK
PGSFDRSIPENNIMRTIIEFLSFLHLKEAGALDRLLDLPAAASSEDIERS
The mature galanin sequence is accentuated in bold.

### Example 3

### Transfection of Mammalian Cells and Analysis of Secreted Neuropeptide

### Transient Transfection Studies

ARPE-19 is a human retinal pigment epithelial cell line (Dunn et al. 1996) grown in DMEM/Nutrient Mix F-12 with Glutamax (Invitrogen, Denmark) supplemented with 10% fetal bovine serum (Sigma-Aldrich, Denmark) at 37° C. and 5% CO₂. Cells were passaged approximately twice a week by trypsinization and reseeding (1:5 split ratio). Cells were seeded in 6-well plates (Corning Costar, Biotech Line, Denmark) at a density of 10⁵ cells/well for transfection studies. The next day, cells were transfected with 3 μg plasmid/well in duplicate wells using Fugene6 (Roche, Germany) according to the manufacturer's specifications. Cell supernatants collected 3 days after transfection were tested for the presence of galanin using a commercial galanin ELISA kit (cat. # S-1210, Bachem).

### Production of Stable Qalanin Clones

ARPE-19 cells were grown as above, except that cells were seeded into T150 culture flasks at 2.4×10⁶ cells/flask. The next day cells were transfected with 10 μg of plasmid DNA using Fugene6 (Roche, Germany) according to the manufacturer's specifications. 418 selection was applied on the cells after 48 hrs. Colonies were picked after appr. 3 weeks and propagated in 48 well plates until at least 70% confluent. Supernatants from the propagated colonies were tested for the presence of galanin using the commercial galanin ELISA mentioned above. The highest producing clone from each galanin construct was further expanded in T150 flasks and 100 ml supernatant was collected for purification and sequence characterisation of secreted galanin as well as a receptor binding test (See description below).

### Receptor Binding Assay

Testing of binding of galanin secreted from galanin-producing cell clones was done using the GalR1Screen Ready Target Assay (Perkin Elmer). The assay is based on competition between the test sample and a radioactive ligand, [¹²⁵I]-Galanin, for binding to the galanin receptor type 1, GalR1. Thus, the degree of displacement of the radioactive ligand [¹²⁵I]-Galanin is measured. Briefly, 12.5 μl binding buffer (50 mM Tris-HCl, pH 7.4, 50 mM MgCl₂, 1 mM EDTA, 0.1% Bacitracin, 0.5% BSA) with or without galanin is first added to flashplates coated with membranes from HEK-293 cells expressing human GalR1 on the surface. Then 12.5 μl [¹²⁵I]-galanin is added and the mixture is incubated at room temperature for 1 hour before counting samples using a Scintillation counter (30 seconds/well) (Perkin Elmer, TriLux counter). No washing step is necessary, since only radioactive [¹²⁵I]-Galanin bound to the receptor will scintillate due to the flash-plate coating.

### Results

### Galanin Secretion

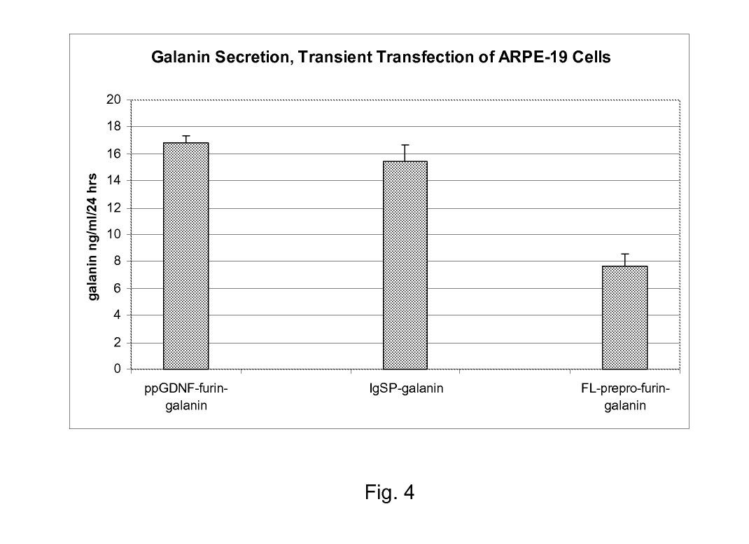

Galanin was detected in supernatants from cells stably transfected with each of the three galanin constructs. From FIG. 4 it is clear that the three different signal peptides (from wt galanin, wt GDNF and IgSP) all lead to secretion of galanin.

### Galanin Receptor Binding

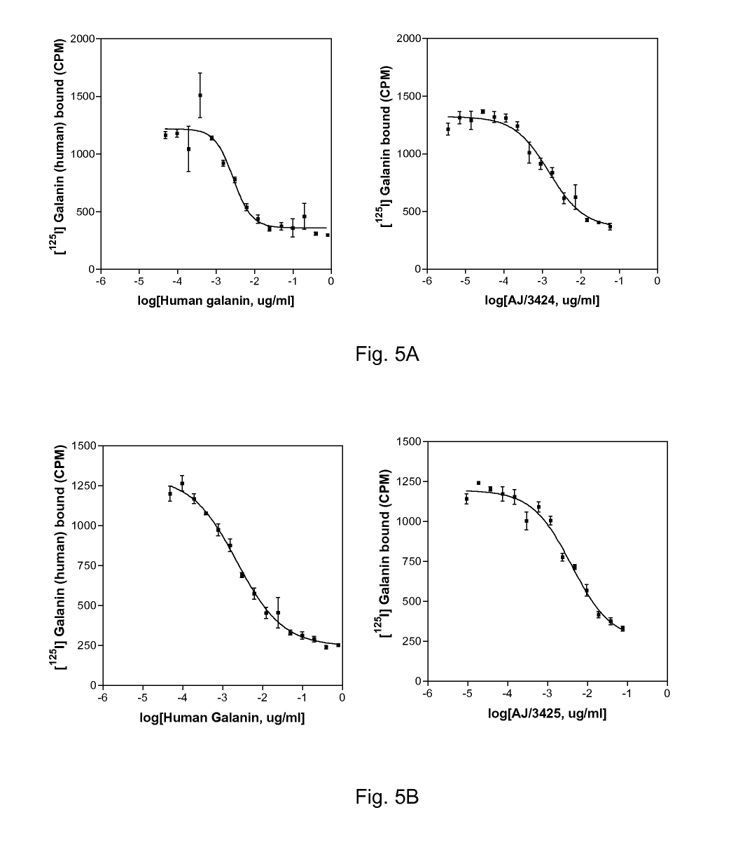

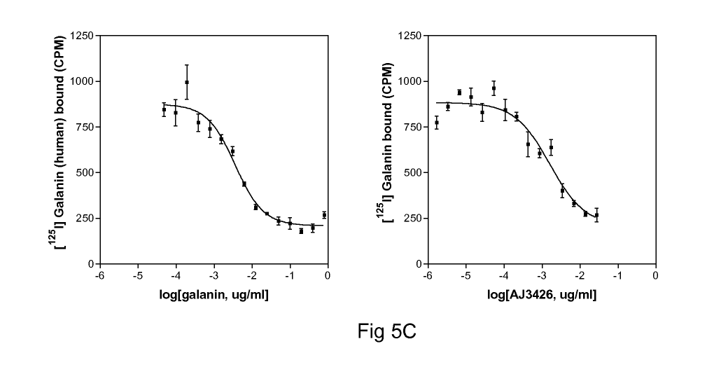

One hundred ml of supernatant from clones stably transfected with each of the three galanin constructs was generated for reversed phase HPLC purification. After first round of purification the concentrated galanin was tested in a receptor binding assay in comparison with recombinant human galanin (see FIGS. 5A, 5B and 5C).

From figures FIG. 5A-5C it is clear, that the binding profile of galanin secreted from ARPE-19 cells stably transfected with the three expression constructs is very similar to the binding profile of recombinant human galanin. Hence, the affinity of galanin derived from the three constructs for galanin receptor 1, GalR1, is as good as recombinant human galanin.

### Protein Purification and Identification of Secreted Galanin

After four rounds of reversed phase HPLC purification of galanin from supernatants of ARPE-19 cells stably transfected with the three galanin constructs, the three samples of galanin could be subjected to sequence characterisation by mass spectrometry.

The purified galanin fractions derived from two constructs (FLprepro-furin-galanin and ppGDNF-furin-galanin) were adjusted to pH 4, centrifuged for 30 min at 10,000 g, and the supernatants filtered consecutively through 1.2 and 0.45 μm filters (Milipore). The filtrates were purified through a number of HPLC steps. For all steps were used gradients from A: 0.1% TFA in H₂O to B: 0.1% in acetonitrile. Four different columns were used in the following order applying gradients of 0.5%/min 1) Vydac C4, 5 μm, 4.6×250 mm, 2) Vydac phenyl, 5 μm, 2.1×150 mm, 3) Vydac C8, 5 μm, 2.1×150 mm, 4) Vydac C18 monomeric, 5 μm, 2.1×150 mm. Finally in step 5) the latter column was employed again using a gradient of 0.2%/min. For step 1) fractions of 1 min were collected, for step 2) and 3) fractions of 0.5 min and for 4) and 5) peak fractions (monitored at 214 nm) were collected manually. For the first four steps the fractions were monitored for immunoreactivity and the fractions containing immunoreactivity were pooled for the next step The immunoreactive fractions from step 4) were analysed by MALDI-TOF mass spectrometry (using α-cyano-4 hydroxycinnamic acid in acetonitrile-methanol (Agilent) as matrix) on an AutoFlex II instrument equipped with Tof-Tof facility (Bruker, Bremen). Although not pure, species with the molecular mass expected for mature galanin were observed: from 676-2: 3155.42 and from 680-42: 3155.52 compared to the theoretical monoisotopic value 3155.55. Tof-Tof analysis of the first peptide, followed by search in the NCBR database using the Mascot search engine (www.matrixscience.com) indicated galanin as the first choice, even if the score was rather low. In an attempt to purify the peptide further step 5) was performed. The fractions containing the 3155-species were analysed by LC-MSMS on a Q-T of 2 (Waters) coupled to an Easy nLC (Proxeon Biosystems) equipped with a Biosphere C18 column, 5 μm, 75 μm×100 mm (NanoSeparations). MSMS was performed during the HPLC run including only the relevant molecular mass and by search as described above only galanin was indicated with a convincing match, this time p<0.05 for the 672-2 construct and slightly less significant for the 680-42 construct.

### Conclusion

All three tested expression constructs thus lead to secretion of correctly processed mature human galanin in high amount.

### Example 4

### Comparative examples

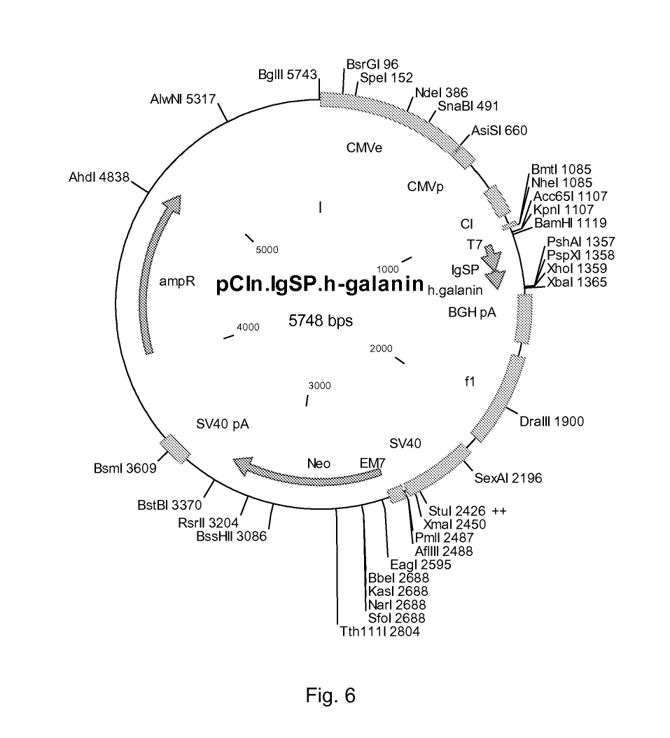

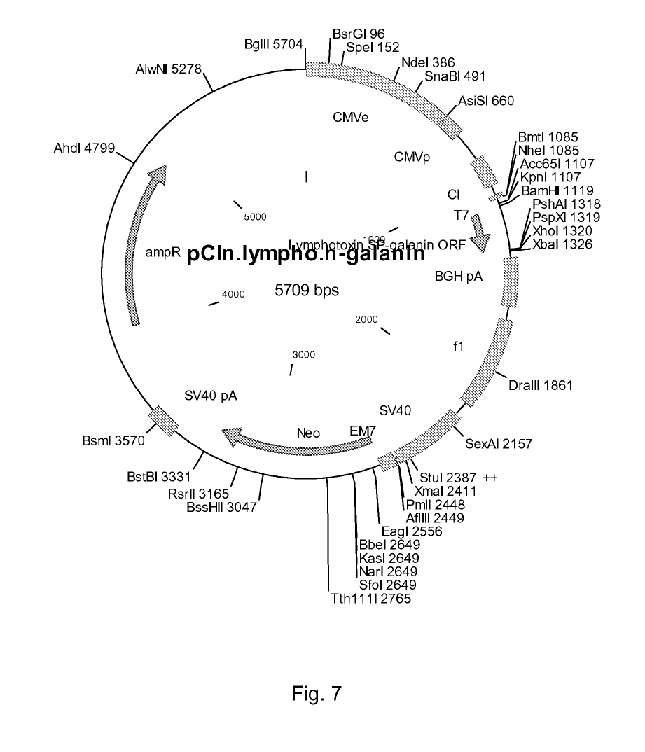

Construction of qalanin expression plasmids for production of wt qalanin by ARPE-19 cells The secretion apparatus of the eukaryotic cell can not secrete proteins with a total size of else than 50-70 amino acids. Due to the small size of mature human galanin (30 amino acids), initial attempts to get galanin secretion from ARPE-19 cells were focused on long signal peptides. Using SignalP (signal peptide cleavage prediciton tool, http://www.cbs.dtu.dk/services/SignalP/), signal peptide-galanin constructs were tested in silico to find the fusions with highest signal peptide cleavage propability. Three constructs were made where mature galanin was fused at its N-terminus to the signal peptides for mouse immunoglobulin heavy chain V-region (IgSP), Lymphotoxin and Semaphorin. The three constructs were made as follows:
1. The IgSP-galanin construct was generated by overlapping PCR.
   In the first amplification step the sequence of mature galanin was PCR amplified from pCMV-SPORT6-hgalanin (obtained from RZPD, clone ID: IRATp970F0849D6) using the primers FLAP-IgSP-mature gala, 5′ (SEQ ID NO 65) (5′-GGTGAATTCGGGCTGGACCCTGAACA GCGCG-3′) and Mature gala-STOP-XhoI 3′ (SEQ ID NO 64) (5′-TATACTCGAGTCAGCTGGTGAGGCCATT CTTGTCGC-3′). In a second PCR reaction the IgSP fragment was amplified from pNUT-IgSP-hCNTF (obtained from Neurotech) using primers IgSPkozak1s+BamHI (SEQ ID NO 67) (5′-TATAGGATCCGCCACCATGAAATGCAGCTGGGTTATC-3′) and IgSP-galanin FLAP as (SEQ ID NO 68) (5′-GGGTCCAGCCCGAATTCACCCCTGTAGAAAG-3′). In the third step the products of step 1 and 2 were combined in a final PCR reaction that generated the IgSP-mature galanin fragment by using equimolar amounts of products of the first two PCR reactions and the primers IgSPkozak1s+BamHI and Mature gala-STOP-XhoI 3′.
   To generate a plasmid-based expression vector the resulting PCR fragment was cloned into pCl digested with BamHI/XhoI. In this vector, the IgSP-mature galanin sequence is placed under transcriptional control of the CMV promoter (see FIG. 6). Furthermore, the vector contains the Neo gene that confers G418 resistance when expressed in mammalian cells.
2. The Lympotoxin-galanin construct was generated by overlapping PCR. In the first amplification step the sequence of mature galanin was PCR amplified from pCMV-SPORT6-hgalanin (obtained from RZPD, clone ID: IRATp970F0849D6) using the primers FLAP-lymphotoxin-mat gala 5′ (SEQ ID NO 69) (5′-GGCCCAGGGGGGCTGGACCCTG AACAGCGC-3′) and Mature gala-STOP-XhoI 3′ (SEQ ID NO 64) (5′-TATACTCGAGTCAGCTGGTGAGGCC ATTCTTGTCGC-3′). In the second step the lymphotoxin signal peptide DNA sequence was made synthetic by annealing adaptors BamHI-Lymphotoxin 5′ Long (SEQ ID NO 70) (5′-CGGGATCCATGACACCACCTGAACGTCTCTTCCTCCCAAGGGTGCGTGG CACCAC CCTACACCTCCTCCTTCTGGG-3′) and Lymphotoxin-mat gala FLAP 3′ (SEQ ID NO 71) (5′-GGGTCC AGCCCCCCTGGGCCCCAGGCAGCAGAACCAGCAGCAGCCCCAGAAGG AGGAGGTGTAG-3′). In a second PCR reaction the products of step 1 and 2 were combined in a final PCR reaction that generated the Lymphtoxin-mature galanin fragment by using equimolar amounts of products of the first two steps and the primers BamHI-Lymphotoxin 5′ Short and Mature gala-STOP-XhoI 3′.
   To generate a plasmid-based expression vector the resulting PCR fragment was cloned into pCl digested with BamHI/XhoI. In this vector, the Lymphotoxin-mature galanin sequence is placed under transcriptional control of the CMV promoter (see FIG. 7). Furthermore, the vector contains the Neo gene that confers G418 resistance when expressed in mammalian cells.
3. The Semaphorin-galanin construct was generated by overlapping PCR. In the first amplification step the sequence of mature galanin was PCR amplified from pCMV-SPORT6-hgalanin (obtained from RZPD, clone ID: IRATp970F0849D6) using the primers FLAP-semaphorin-mat gala 5′ (#1680) (SEQ ID NO 72): 5′-GACCTGGGCGGGCT GGACCCTGAACAGCGC-3′ and Mature gala-STOP-XhoI 3′ (SEQ ID NO 64) (5′-TATACTCGAGTCA GCTGGTGAGGCC ATTCTTGTCGC-3′). In the second step the semaphorin signal peptide DNA sequence was made synthetic by annealing adaptors BamHI-Semaphorin 5′ Long (SEQ ID NO 73) (5′-CGGGATCCATGGGCCTGAGGAGCTGGCTCGCCGCCCCATG GGGCGCGCTGCCGCCTCGGCCACCGCTGCTGCTGCTCCTGCTGC-3′) and Semaphorin-mat gala FLAP 3′ (SEQ ID NO 74) (5′-GGGTCCAGCCCGCCCAGGTCGGAGGCGGCGGCTGCAGCAGGAG CAGCAGCAGCAGGAGCAGCAGCAGC-3′). In a second PCR reaction the products of step 1 and 2 were combined in a final PCR reaction that generated the Lymphtoxin-mature galanin fragment by using equimolar amounts of products of the first two steps and the primers BamHI-Lymphotoxin 5′ Short and Mature gala-STOP-XhoI 3′.

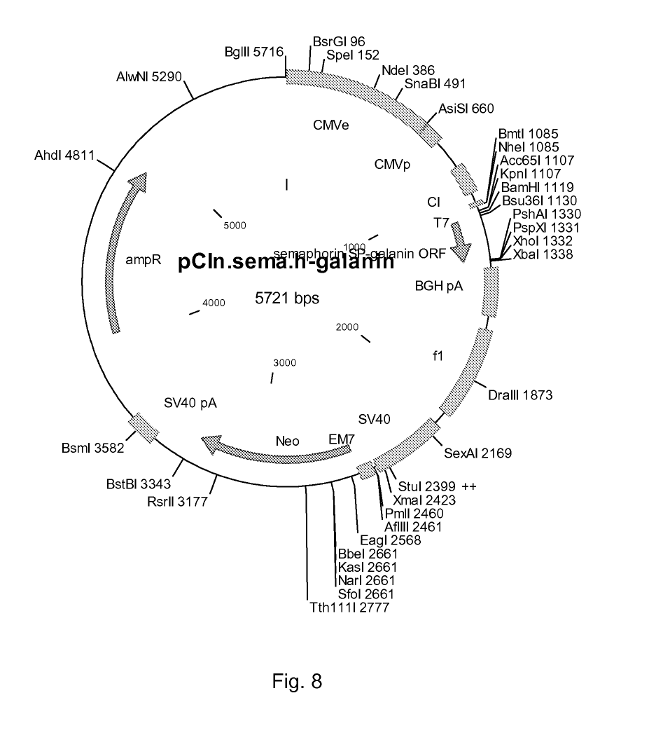

To generate a plasmid-based expression vector the resulting PCR fragment was cloned into pCl digested with BamHI/XhoI. In this vector, the Semaphorin-mature galanin sequence is placed under transcriptional control of the CMV promoter (see FIG. 8). Furthermore, the vector contains the Neo gene that confers G418 resistance when expressed in mammalian cells. Expression test of the three constructs:

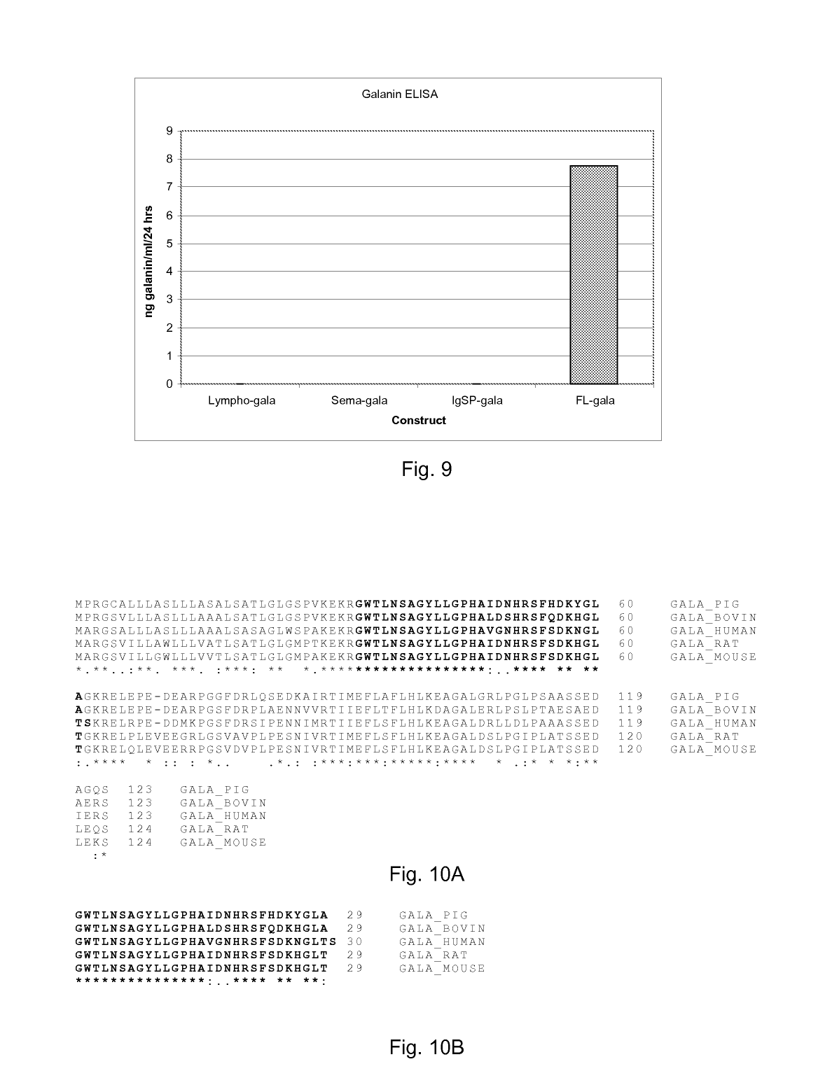

A galanin ELISA kit (Bachem cat #S-1210) was used to test for galanin secreted from ARPE-19 cells transiently transfected with each of the three above mentioned constructs. The results are shown in FIG. 9.

It is clear from FIG. 9 that a long signal peptide fused to a short mature peptide does not lead to secretion.

Expression of full length wild type galanin (pre-pro-mature-C-terminal peptide) leads to secretion of galanin. However, galanin secreted using this construct also contained the galanin pro-peptide due to the presence of a sub-optimal furin cleavage sequence. Similarly, expression of a construct consisting of ppGDNF fused to mature galanin, lead to secretion, but the propeptide was not processed.

Protein sequences of the three galanin constructs described above:

Lymphotoxin-galanin
(SEQ ID NO 8)
MTPPERLFLPRVRGTTLHLLLLGLLLVLLPGAQGGWTLNSAGYLLGPHA
VGNHRSFSDKNGLTS
Semaphorin-galanin
(SEQ ID NO 9)
MGLRSWLAAPWGALPPRPPLLLLLLLLLLLQPPPPTWAGWTLNSAGYLL
GPHAVGNHRSFSDKNGLTS
IgSP-galanin
(SEQ ID NO 10)
MKCSWVIFFLMAVVTGVNSGWTLNSAGYLLGPHAVGNHRSFSDKNGLTS
Underlined = signal peptide
ppGalanin
(SEQ ID NO 11)
MARGSALLLASLLLAAALSASAGLWSPAKEKRGWTLNSAGYLLGPHAVG
NHRSFSDKNGLTSKRELRPEDDMKPGSFDRSIPENNIMRTIIEFLSFLH
LKEAGALDRLLDLPAAASSEDIERS
Wild-type human galanin translated peptide.
Mature galanin is shown in bold
ppGDNF-galanin
(SEQ ID NO 12)
MKLWDVVAVCLVLLHTASAFPLPAGKRPPEAPAEDRSLGRRRAPFALSS
DSNMPEDYPDQFDDVMDFIQATIKRLKRGWTLNSAGYLLGPHAVGNHRS
FSDKNGLTS
ppGDNF fused to mature human galanin peptide.
The mature peptide is shown in bold.

### Example 5

### Preparation of Devices for CNS Use

Devices are fabricated from polysulphone (PS), or polyether sulfone (PES) or an equivalent polymer hollow fiber membrane with an outside diameter of 800-1000 μm and a wall thickness of approximately 100 μm. A scaffolding material consisting of polyvinyl alcohol (PVA) sponge, polyethylene (PET) yarn or similar material inserted into the membrane fiber cavity ensures proper cell distribution and attachment of the cells. Finally, a tether fabricated from polyurethane (PU) or an equivalent material fixed to the device end provides a means for device retrieval post-implantation.

Devices used for pre-clinical testing (in rats) are approximately 5-7 mm long. Devices contemplated for implantation into human brains are approximately 5-20 mm long.

Cellular loading occurs through a hub segment and port attached to the hollow fiber device at the end distal to the tether. Neuropeptide cells prepared as a single-cell suspension are infused into the port, the hub segment is retrieved and the infusion hole is sealed with glue. For each mm length of the devices, approximately 10,000 neuropeptide-expressing cells are loaded. The devices are maintained in media until use.

Devices for implantation into rat brains were made with the following materials: Membrane: PS device: Polysulphone hollow fiber membrane (PS90/700 from Minntech Corp, Minneapolis, Minn., USA), with a 90 kDA molecular weight cutoff. Dimensions: 700 μm+/−50 μm inner diameter, 100 μm+/−20 μm wall. PES device: Polyethersulfone: PES5 from Akzo Nobel with a 280 kDa molecular weight cutoff. Dimensions: 520 μm+/−50 μm inner diameter, 100 μm+/−20 μm wall. Hollow fibers were cut to lengths of approximately 5 mm (PS device) and 7 mm (PES device).

Foam: PS device: PVA foam, product no. 160 LD from Hydrofera Inc, Cleveland, Ohio, USA. PES device: Clinicel sponge from M-PACT, Eudora, Kansas, USA. The PVA foam was cut to fit the inner diameter of the hollow fiber.
Load tube: Perfluoroalkoxy copolymer. Dimensions: PS device: 0.0037″+/−0.0005″ ID; 0.005″+/−0.001″ wall. PES device: 410 μm+/−50 μm ID; 45 μm+/−5 μm wall, both from Zeus Industrial Products, Orangeburg, S.C., USA. The load tube is glued to the hollow fiber in one end and to the hub in the other end.
Hub: Product no P/N 02030200 Rev 1, from Abtec, Bristol, Pa., USA. Glue for gluing load tube to hub: Dymax 201-CTH (Diatom, Hvidovre, Denmark).
Glue for hollow fiber: PS device: Dymax 1181-M. PES device: Dymax 1188-M. Devices were assembled in a controlled environment, packaged in Falcon 15 mL polypropylene test tubes (Becton Dickinson, Cat #352096) and sterilised by exposure to ethylene oxide prior to filling with cells.

### Example 6

### Intrathecal Implantation of Devices

Intrathecal implantation can be accomplished along the spinal canal, preferably at the lumbar level below the conus medullaris in e.g. human beings. A small incision is made at the lumbar level, and a spinal needle is used to enter the intrathecal space. After CSF flow has been established, a guide wire is inserted into the intrathecal space and a dilator system is used to enter the space. The guidewire is withdrawn and the encapsulated device inserted into the space so that the active part is completely enclosed in the CSF compartment. The tether is secured to the lumbar fascia by a non-resorbable suture and preferably using a securing clip. The skin is closed using standard surgical procedures.

### Example 7

### Implantation in the Human Striatal Structures

Under general anesthesia or local anesthesia and sedation, a neurosurgical stereotactic frame is secured to the patient's head. A fiducial box and subsequent MRI imaging is applied to determine the anatomical area and implantation coordinates. The implantation can also be guided by diffusion tensor imaging and dose mapping, utilising custom software and navigational equipment supplied by BrainLAB AG. The patient is next brought to the operating room where he/she is prepped and draped. Based on the stereotactic image data a small skin incision is made frontolaterally and a small burrhole made through the skull. The dura and underlying meninges are penetrated by incision and a guide cannula with a trochar is inserted into the putamen and caudate nucleus target area. The trocar is removed and the device is slided into position. The guide is removed and the device tether secured to the skull with a titanium plate or custom retaining clip. One or more devices may be inserted into the same structure. The skin is sutured closed with interrupted 3-0 Nylon suture. The procedure is repeated on the opposite side.

### Example 8

### Construction of Galanin Expression Plasmids and Sub-Cloning of the Expression Cassettes into the Substrate Vector of the Sleeping Beauty Transposase for Production of wt Galanin by Mammalian Cells

1) The galanin constructs were made as follows: IgSP-deltaprepro-galanin was generated by overlapping PCR. In the first amplification step the galanin sequence coding for mature galanin and the C-terminal peptide with 10 bp IgSP FLAP (IgSP=mouse Ig heavy chain gene V-region signal peptide sequence) was PCR amplified from the pCMV-SPORT6-hgalanin plasmid (obtained from RZPD Berlin, Germany, clone ID: IRATp970F0849D6) using primers FLAP-IgSP-mature gala, 5′ (SEQ ID NO 65) (5′-GGTGAATTCGGGCTGGACCCTGAACAGCGCG-3′) and Deltaprepro-galanin-XhoI 3′ (SEQ ID NO 66) (5′-TATACTCGAGCAGGAATGGCTGACTCTGCATAAATTGGCC-3′). In a second PCR reaction a fragment containing the full length IgSP sequence with a 10 bp galanin FLAP from the 5′ end of mature galanin was amplified from pNUT-IgSP-hCNTF (U.S. Pat. No. 6,361,771) using primers IgSPkozak1s+BamHI (SEQ ID NO 67) (5′-TATAGGATCCGCCACCATGAAATGCAGCTGGGTTATC-3′) and IgSP-galanin FLAP as (SEQ ID NO 68) (5′-GGGTCCAGCCCGAATTCACCCCTGTAGAAAG-3′). In the third step the products of step 1 and 2 were combined in a final PCR reaction that generated the IgSP-deltaprepro-galanin fragment by using equimolar amounts of products of the first two PCR reactions and the primers IgSPkozak1s+BamHI and Deltaprepro-galanin-XhoI 3′.
To generate a plasmid-based expression vector the resulting PCR fragment was cloned into pCAn digested with BamHI/XhoI. The pCAn vector is derived from pcDNA3.1 (Invitrogen). The CMV promoter was removed from pcDNA3.1 and replaced with the human CMV enhancer/chicken beta-actin (CAG) promoter and first intron. Furthermore, the vector contains the Neo gene that confers G418 resistance when expressed in mammalian cells. The IgSP-deltaprepro-galanin fragment expression cassette (i.e. including the CAG promoter as well as neomycin resistance expression cassette) was then sub-cloned from the pCAn vector into plasmid pT2BH. pT2BH is the substrate vector for the transposase Sleeping Beauty (Ivics et al., Cell, 91: 501-10 (1997)). The sub-cloning was done by first digesting pT2BH with BglII and EcoRV. The pCAn-IgSP-deltaprepro-galanin vector was then digested with BsmBI followed by fill-in reaction with Klenow large fragment polymerase. The blunted, opened vector was then digested with BglII to create a semi-blunt IgSP-deltaprepro-galanin+neomycn resistance expression cassette fragment, which was cloned into the BgIII-EcoRV-digested pT2BH vector.

Sequences from the constructs are shown in Example 9.

### Example 9

### Sequences of the Constructs Described in Example 8

IgSP-deltaprepro-galanin nucleotide sequence present in constructs pCAn.IgSP-deltaprepro-galanin and pT2.CAn.IgSP-deltaprepro-galanin (SEQ ID NO 5)

IgSP-deltaprepro-galanin is the mouse Ig heavy chain gene V-region signal peptide (GenBAnk ID: M18950) fused to human galanin devoid of the prepro sequence but including the C-terminal tail. Note that the IgSP-deltaprepro-galanin sequence contains an intron. Translation of the IgSP transcript results in a polypeptide having SEQ ID NO 7

IR/DR (L) left hand (complementary strand) Sleeping Beauty (SB)
substrate sequence present in pT2-derived constructs
(SEQ ID NO 22)
CAGTTGAAGTCGGAAGTTTACATACACTTAAGTTGGAGTCATTAAAACTCGTTTTTCAACTACTC
CACAAATTTCTTGTTAACAAACAATAGTTTTGGCAAGTCAGTTAGGACATCTACTTTGTGCATGA
CACAAGTCATTTTTCCAACAATTGTTTACAGACAGATTATTTCACTTATAATTCACTGTATCACA
ATTCCAGTGGGTCAGAAGTTTACATACACTAA
IR/DR (R) right hand SB substrate sequence present in
pT2-derived constructs
(SEQ ID NO 23)
TTGAGTGTATGTAAACTTCTGACCCACTGGGAATGTGATGAAAGAAATAAAAGCTGAAATGAATC
ATTCTCTCTACTATTATTCTGATATTTCACATTCTTAAAATAAAGTGGTGATCCTAACTGACCTA
AGACAGGGAATTTTTACTAGGATTAAATGTCAGGAATTGTGAAAAAGTGAGTTTAAATGTATTTG
GCTAAGGTGTATGTAAACTTCCGACTTCAACTG
Protein sequence of SB transposase SB10
(wt Sleeping Beauty transposase)
(SEQ ID NO 24)
MGKSKEISQD LRKKIVDLHK SGSSLGAISK RLKVPRSSVQ TIVRKYKHHG TTQPSYRSGR
RRVLSPRDER TLVRKVQINP RTTAKDLVKM LEETGTKVSI STVKRVLYRH NLKGRSARKK
PLLQNRHKKA RLRFATAHGD KDRTFWRNVL WSDETKIELF GHNDHRYVWR KKGEACKPKN
TIPTVKHGGG SIMLWGCFAA GGTGALHKID GIMRKENYVD ILKQHLKTSV RKLKLGRKWV
FQMDNDPKHT SKVVAKWLKD NKVKVLEWPS QSPDLNPIEN LWAELKKRVR ARRPTNLTQL
HQLCQEEWAK IHPTYCGKLV EGYPKRLTQV KQFKGNATKY
Protein sequence of hyperactive SB transposase SB100x
(SEQ ID NO 25)
MGKSKEISQD LRKRIVDLHK SGSSLGAISK RLAVPRSSVQ TIVRKYKHHG TTQPSYRSGR
RRVLSPRDER TLVRKVQINP RTTAKDLVKM LEETGTKVSI STVKRVLYRH NLKGHSARKK
PLLQNRHKKA RLRFATAHGD KDRTFWRNVL WSDETKIELF GHNDHRYVWR KKGEACKPKN
TIPTVKHGGG SIMLWGCFAA GGTGALHKID GIMDAVQYVD ILKQHLKTSV RKLKLGRKWV
FQHDNDPKHT SKVVAKWLKD NKVKVLEWPS QSPDLNPIEN LWAELKKRVR ARRPTNLTQL
HQLCQEEWAK IHPNYCGKLV EGYPKRLTQV KQFKGNATKY
Mutations are accentuated in bold and underlined.
Protein sequence of hyperactive SB transposase SB80x
(SEQ ID NO 26)
MGKSKEISQD LRKRIVDLHK SGSSLGAISK RLAVPRSSVQ TIVRKYKHHG TTQPSYRSGR
RRVLSPRDER TLVRKVQINP RTTAKDLVKM LEETGTKVSI STVKRVLYRH NLKGHSARKK
PLLQNRHKKA RLRFATAHGD KDRTFWRNVL WSDETKIELF GHNDHRYVWR KKGEACKPKN
TIPTVKHGGG SIMLWGCFAA GGTGALHKID GIMDAVQYVD ILKQHLKTSV RKLKLGRKWV
FQHDNDPKHT SKVVAKWLKD NKVKVLEWPS QSPDLNPIEN LWAELKKRVR ARRPTNLTQL
HQLCQEEWAK IHPNYCEKLV EGYPKRLTQV KQFKGNATKY
Mutations are accentuated in bold and underlined.

### Example 10

### Generation of Stable Galanin-Secreting Mammalian Cells and Comparison of Levels of Secreted Neuropeptides

### Production of Stable Cell Lines in ARPE-19 Cells Using the Constructs Described in Example 8

ARPE-19 is a human retinal pigment epithelial cell line (Dunn et al. 1996) grown in DMEM/Nutrient Mix F-12 with Glutamax (Invitrogen, Denmark) supplemented with 10% fetal bovine serum (Sigma-Aldrich, Denmark) at 37° C. and 5% CO₂. Cells were passaged approximately twice a week by trypsinization and reseeding (1:5 split ratio). Cells were seeded in T150 flasks (Corning Costar, Biotech Line, Denmark) at a density of 2.4×10⁶ cells/flask for transfection studies. The next day, cells in each flask were co-transfected with pT2 SB substrate vector containing galanin expression cassettes and the SB-100× hyperactive transposase expression vector using either a 3:1 ratio (7.5 μg pT2 vector and 2.5 μg SB-100×) or a 10:1 ratio (9 μg pT2 vector and 0.9 μg SB-100×) using Fugene6 (Roche, Germany) according to the manufacturer's specifications. To select for stable transfectants, 48 hours post-transfection 800 μg/ml G418 was added to the culture medium. When clones appeared were well-defined and separated from each other, approximately 200 clones from each construct were picked and transferred to 48 well plates. When confluent in these plates, galanin clones were tested for the presence of galanin using a commercial galanin ELISA kit (cat. # S-1210, Bachem). The highest producing clones were further expanded in T150 flasks and aliquots were frozen in liquid N₂.

### Results

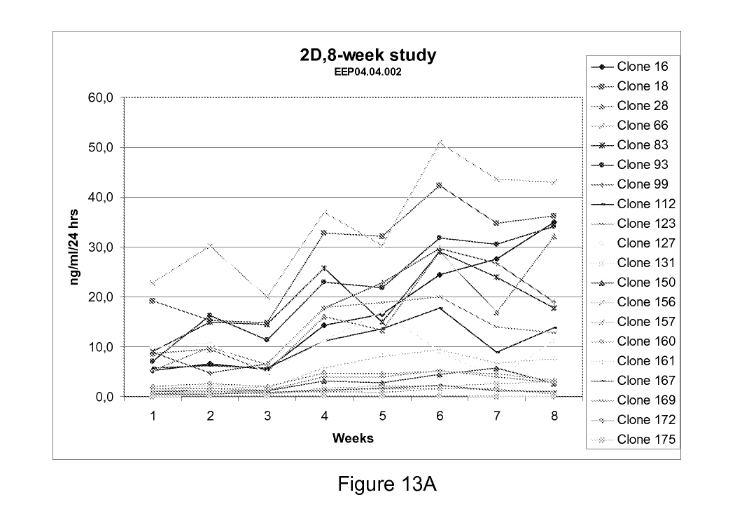

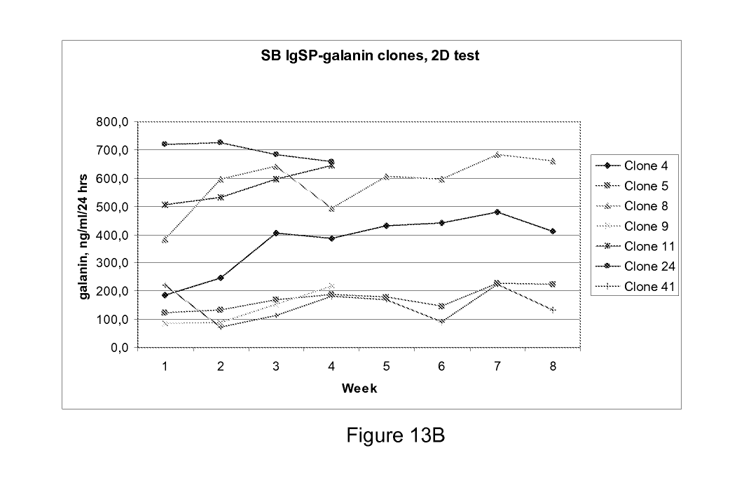

Galanin secretion from pCAn-based and pT2-based clones in vitro The best galanin clones were subjected to expression stability studies in culture for up to 8 weeks (2D studies). From FIG. 13 it is clear that clones generated using the SB transposase system secrete surprisingly large amounts of factor as compared to clones generated by standard transfection.

Galanin Secretion from pCAn-Based and pT2-Based Clones In Vivo

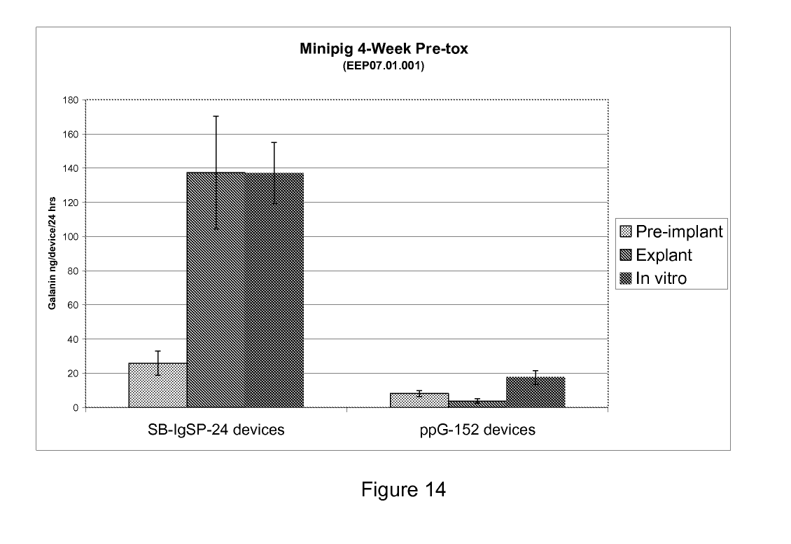

The most stable of galanin high (SB-IgSP-24) and low (ppG-152) producer clones from the 2D-study were tested for expression stability in vivo in the Goettingen minipig model. The clones were encapsulated using NsGene's proprietary Encapsulated Cell (EC) Biodelivery technology. In short, 14 mm polyether-sulphone (PES) membranes with a molecular weight cut-off (MWCO) of 280 kD were filled with 250,000 cells/device. Cells were allowed to settle and propagate on devices for 2 weeks before implantation in the hippocampus of the pigs. Devices were explanted after 4 weeks. FIG. 14 shows secreted galanin levels from devices before implanation compared to explanted devices and devices run in parallel in vitro. It is clear that the secretion level of galanin from the clone, SB-IgSP-24, produced using the SB technology, is unexpectedly large as compared to the clone, ppG-152, generated using standard transfection techniques (explant levels: appr. 150 ng/device/24 hrs vs. 5 ng/device/24 hrs).

### Example 11

### Determination of Transgene Copy Numbers in Cell Lines Generated Using a Tranposase System

The technique used for this determination is called transposon display (Wicks et al., Dev. Biol. 221: 295-307 (2000)), which is a derivative from the vectorette method (Hui et al., Cell. Mol. Life. Sci. 54: 1403-11 (1998)).

The method in brief: 1) Genomic DNA is prepared from the cell line. 2) The genomic DNA is digested with a restriction enzyme to fragment the chromosomes. 3) A, so called, vectorette linker/cassette of appr. 500 bp—with an overhang matching the overhangs of the genomic DNA created by the restriction enzyme—is ligated to the digested genomic DNA. The vectorette linker contains a central appr. 50 bp mismatch region. 4) A two step vectorette PCR is carried out using a primer annealing to one strand of the vectorette and another primer annealing to a sequence in the transposon. Due to the mismatch region in the vectorette linker, only fragments containing a vectorette linker ligated to a digested genomic DNA fragment containing a copy of the transgene (surrounded by transposase substrate sequences) will be amplified (see FIG. 1 from Hui et al., Cell. Mol. Life. Sci. 54: 1403-11 (1998).

Using this method on galanin secreting cell lines created with the Sleeping Beauty transposon system, a good correlation between copy number and secretion levels of the transgene factor was found (see table 2). High producer SB clones typically have 1-18 transgene copies of Galanin. Thus, there is to some extent a correlation between number of transgene copies and the observed improvement in factor secretion levels of SB-clones. However, when comparing the factor secretion levels of SB-clones with one transgene copy and the clones generated using standard transfection techniques also containing one copy of the transgene, it is clear that the number of transgene copies is not the only thing determining secretion level and the SB-clones do have a factor secretion level that is higher and more stable than would normally be expected.

### CONCLUSION

It is clear from the above tests of SB-derived clones versus clones derived using standard transfection techniques, that the SB system is capable of boosting secretion of the transgene more than would be expected from an increase of 3-5 in transgene copy number in the host cell.

| Galanin | Galanin | |
|---|---|---|
| clones | ng/ml/24 hrs | Copy number |
| SB-IgSP4 | 384 | 5 |
| SB-IgSP5 | 187 | 1 |
| SB-IgSP8 | 600 | 12-13 |
| SB-IgSP11 | 644 | 7-9 |
| SB-IgSP24 | 657 | 9 |
| SB-IgSP41 | 182 | 2-3 |

TABLE 2
Comparison of factor secretion levels over a wide range with copy
numbers from clones secreting galanin generated using the
Sleeping Beauty transposon system.
Sequences

| SEQ ID | | |
|---|---|---|
| NO | Type | Description |
| 1 | N | Full length prepro-furin-galanin |
| 2 | P | Full length prepro-furin-galanin |
| 3 | N | ppGDNF-furin-galanin (delta C) |
| 4 | P | ppGDNF-furin-galanin (delta C) |
| 5 | N | IgSP-galanin |
| 6 | N | IgSP-galanin-spliced transcript |
| 7 | P | IgSP-galanin |
| 8 | P | Lymphotoxin-galanin (delta C) |
| 9 | P | Semaphorin-galanin (delta C) |
| 10 | P | IgSP-galanin (delta C) |
| 11 | P | Prepro-galanin (delta C) |
| 12 | P | PreproGDNF-galanin (delta C) |
| 13 | P | Human IgSP |
| 14 | P | Rhesus IgSP |
| 15 | P | Marmoset IgSP |
| 16 | P | Mouse IgSP |
| 17 | P | Pig IgSP |
| 18 | P | Rat IgSP |
| 19 | P | Human Growth Hormone SP |
| 20 | P | Rat Albumin SP |
| 21 | P | Modified rat albumin SP |
| 22 | N | IR/DR left hand (complementary strand) Sleeping Beauty |
| | | substrate sequence present in pT2 derived constructs |
| 23 | N | IR/DR right hand Sleeping Beauty substrate sequence |
| | | present in pT2 derived constructs |
| 24 | P | Sleeping Beauty transposase SB10 (wild type Sleeping |
| | | Beauty transposase) |
| 25 | P | Protein sequence of hyperactive SB transposase (SB100X) |
| 26 | P | Protein sequence of hyperactive SB transposase (SB80X) |
| 27 | P | Pig galanin precursor |
| 28 | P | Bovine galanin precursor |
| 29 | P | Human galanin precursor |
| 30 | P | Rat galanin precursor |
| 31 | P | Mouse galanin precursor |
| 32 | P | Pig galanin mature |
| 33 | P | Bovine galanin mature |
| 34 | P | Human galanin mature |
| 35 | P | Rat galanin mature |
| 36 | P | Mouse galanin mature |
| 37 | P | Rat orexin precursor |
| 38 | P | Mouse orexin precursor |
| 39 | P | Human orexin precursor |
| 40 | P | Pig orexin precursor |
| 41 | P | Rat orexin A |
| 42 | P | Mouse orexin A |
| 43 | P | Human orexin A |
| 44 | P | Pig orexin A |
| 45 | P | Rat orexin B |
| 46 | P | Mouse orexin B |
| 47 | P | Human orexin B |
| 48 | P | Pig orexin B |
| 49 | P | Rhesus NPY precursor |
| 50 | P | Human NPY precursor |
| 51 | P | Rat NPY precursor |
| 52 | P | Mouse NPY precursor |
| 53 | P | Rhesus NPY mature |
| 54 | P | Human NPY mature |
| 55 | P | Rat NPY mature |
| 56 | P | Mouse NPY mature |
| 57 | N | Primer BamHI-preprogalanin 5′ |
| 58 | N | Primer Galanin prepro furin FLAP as |
| 59 | N | Primer Furin FLAP-mature galanin |
| 60 | N | Primer Preprogalanin-Xhol 3′ |
| 61 | N | Primer Bam-5′ preproGDNF |
| 62 | N | Primer ppGDNF-furin-gaIFLAP |
| 63 | N | Primer 5′ gal-furin-ppGDNF FLAP s |
| 64 | N | Primer Mature gala-STOP-Xhol 3′ |
| 65 | N | Primer FLAP-IgSP-mature gala 5′ |
| 66 | N | Primer Deltaprepro-galanin-Xhol 3′ |
| 67 | N | Primer IgSPkozak1s + BamHI |
| 68 | N | Primer IgSP-galanin FLAP as |
| 69 | N | Primer FLAP-lymphotoxin-mat gala 5′ |
| 70 | N | Primer BamHI-Lymphotoxin 5′ Long |
| 71 | N | Primer Lymphotoxin-mat gala FLAP 3′ |
| 72 | N | Primer FLAP-semaphorin-mat gala 5′ |
| 73 | N | Primer BamHI-Semaphorin 5′ Long |
| 74 | N | Primer Semaphorin-mat gala FLAP 3′ |

1-98. (canceled)

99. An expression construct for expression in a mammalian cell, the expression construct coding for a heterologous polypeptide comprising at least 51 amino acids, said heterologous polypeptide comprising from the N-terminal to the C-terminal a mammalian signal peptide, a pro-peptide, and a neuropeptide, wherein said neuropeptide is cleavable from the pro-peptide by furin at a furin-site, and wherein said furin-site is optimal for cleavage.

100. The expression construct of claim 99, wherein the construct additionally encodes a C-terminal peptide.

101. The expression construct of claim 99, wherein the heterologous polypeptide following cleavage of the signal peptide comprises at least 35 amino acids.

102. The expression construct of claim 99, wherein said heterologous polypeptide comprises less than 200 amino acids.

103. The expression construct of claim 99, wherein said heterologous polypeptide comprises from 51 to 150 amino acids.

104. The expression construct of claim 99, wherein the pre-pro-region and the neuropeptide are heterologous with respect to one another.

105. The expression construct of claim 99, wherein the signal peptide and the pro-peptide are derived from the same pre-pro-protein precursor.

106. The expression construct of claim 105, wherein the precursor protein is galanin.

107. The expression construct of claim 105, wherein the precursor protein is a neurotrophic factor.

108. The expression construct of claim 99, wherein the furin site comprises the following amino acid sequence: P₆P₅P₄P₃P₂P₁↓P₁′P₂′, wherein cleavage takes place between P₁ and P₁′, and wherein P₆P₅P₄P₃P₂P₁↓P₁′P₂′ is R-X-R-X-[K/R]-R-↓X-X, wherein X is any amino acid.

109. The expression construct of claim 108, wherein P₆P₅P₄P₃P₂P₁↓P₁′P₂′ is R-X-R-K-K-R-↓X-X or R-X-R-T-K-R-↓X-X

110. The expression construct of claim 108, wherein P₁′ is neither I, nor L, nor V.

111. The expression construct of claim 108, wherein P₂′ is neither R nor K.

112. The expression construct according to claim 99, wherein the neuropeptide is selected from the group consisting of galanin, neuropeptide Y, orexin A, orexin B, enkephalin, somatostatin 14, somatostatin 28, vasoactive intestinal peptide, intestinal peptide PHV-42, intestinal peptide PHV-27, substance P, neurotensin, cholecystokinin 58, cholecystokinin 39, cholecystokinin 33, cholecystokinin 25, cholecystokinin 18, cholecystokinin 12, cholecystokinin 8, cholecystokinin 7, cholecystokinin 5, substance P, neuropeptide K, neuropeptide gamma, neurokinin A and TRH.

113. The expression construct of claim 112, wherein the neuropeptide is selected from the group consisting of galanin, neuropeptide Y, orexin A, and orexin B.

114. The expression construct of claim 99, wherein the heterologous polypeptide comprises GDNF pre-pro-region with an optimal furin site, linked to galanin.

115. The expression construct of claim 99, wherein the heterologous polypeptide comprises galanin pre-pro-region with an optimal furin site, linked to galanin mature peptide and including galanin C-terminal peptide.

116. An expression construct for expression in a mammalian cell, the expression construct coding for a heterologous polypeptide comprising at least 80 amino acids, said heterologous polypeptide comprising an N-terminal signal peptide linked to a neuropeptide, the neuropeptide being linked to a C-terminal peptide, wherein the native (wildtype) human cDNA coding for said neuropeptide additionally comprises a sequence coding for a pro-peptide, between the signal peptide and the neuropeptide.

117. The expression construct of claim 116, wherein the signal peptide is a heterologous signal peptide.

118. The expression construct of claim 116, wherein the signal peptide is an Immunoglobulin Signal Peptide.

119. The expression construct according to claim 116, wherein the neuropeptide is selected from the group consisting of galanin, cholocystokinin, Neurotensin, substance P, neuropeptide K, neutopeptide gamma, Neurokinin A, vasoactive intestinal peptide and orexin-B,

120. The expression construct of claim 119, wherein the neuropeptide is galanin.

121. The expression construct of claim 116, comprising an immunoglobulin signal peptide linked to mature galanin, linked to galanin C-terminal peptide (SEQ ID NO 7).

122. The expression construct of claim 116, wherein the coding sequence is codon optimised for expression in a host cell, preferably for expression in a human host cell.

123. The expression construct of claim 116, wherein the expression construct is part of a mammalian plasmid expression vector.

124. The expression construct of claim 116, wherein the neuropeptide comprises between 10 and 50 amino acids.

125. An isolated host cell transformed or transduced with the expression construct of claim 99.

126. The host cell of claim 125, being selected from the group consisting of mammalian cells.

127. The host cell of claim 125, being selected from the group consisting of human cells

128. The host cell of claim 125, being selected from the group consisting of immortalised retinal pigmented epithelial cells.

129. The host cell of claim 125, being selected from the group consisting of CHO, CHO-K1, HEI193T, HEK293, COS, PC12, HiB5, RN33b and BHK cells

130. An implantable biocompatible cell device, the device comprising:
i) a semipermeable membrane permitting the diffusion of a neuropeptide as defined by claim 99 and/or a virus vector; and
ii) an inner core comprising a composition of cells transformed or transduced with the expression construct of claim 99.

131. The device of claim 130, wherein the semipermeable membrane is immunoisolatory.

132. The device of claim 130, wherein all cells in the device are derived from the same cell line.

133. The device of claim 130, wherein the expression construct is a plasmid.

134. The device of claim 130, wherein the device is capable of secreting in excess of 1 ng neuropeptide/24 hours.

135. The device of claim 130, comprising between 10,000 and 250,000 cells per μL of device.

136. The device of claim 130, having a volume of at least 0.5 μL.

137. The device of claim 130, containing substantially less than 10⁴ cells.

138. The device of claim 137, having a diameter of less than 250 μm.


SEQUENCE LISTING (74 sequences)

SEQ ID NO: 1 - DNA, 372 bp, Homo sapiens
  CDS (1)..(372)
atggcccgaggcagcgccctccttctcgcctccctcctcctcgccgcggccctttctgcctctgcggggctctggtcgccgtcaagaaaaaaacgaggctggaccctgaacagcgcgggctacctgctgggcccacatgccgttggcaaccacaggtcattcagcgacaagaatggcctcaccagcaagcgggagctgcggcccgaagatgacatgaaaccaggaagctttgacaggtccatacctgaaaacaatatcatgcgcacaatcattgagtttctgtctttcttgcatctcaaagaggccggtgccctcgaccgcctcctggatctccccgccgcagcctcctcagaagacatcgagcggtcctga

SEQ ID NO: 2 - protein, 123 aa, Homo sapiens
MARGSALLLASLLLAAALSASAGLWSPSRKKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 3 - DNA, 324 bp, Homo sapiens
  CDS (1)..(324)
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccacccgttcaagaaaaaaaaggggctggaccctgaacagcgcgggctacctgctgggcccacatgccgttggcaaccacaggtcattcagcgacaagaatggcctcaccagctga

SEQ ID NO: 4 - protein, 107 aa, Homo sapiens
MKLWDVVAVCLVLLHTASAFPLPAGKRPPEAPAEDRSLGRRRAPFALSSDSNMPEDYPDQFDDVMDFIQATRSRKKRGWTLNSAGYLLGPHAVGNHRSFSDKNGLTS

SEQ ID NO: 5 - DNA, 412 bp, Homo sapiens
atgaaatgcagctgggttatcttcttcctgatggcagtggttacaggtaaggggctcccaagtcccaaacttgagggtccataaactctgtgacagtggcaatcactttgcctttctttctacaggggtgaattcgggctggaccctgaacagcgcgggctacctgctgggcccacatgccgttggcaaccacaggtcattcagcgacaagaatggcctcaccagcaagcgggagctgcggcccgaagatgacatgaaaccaggaagctttgacaggtccatacctgaaaacaatatcatgcgcacaatcattgagtttctgtctttcttgcatctcaaagaggccggtgccctcgaccgcctcctggatctccccgccgcagcctcctcagaagacatcgagcggtcctga

SEQ ID NO: 6 - DNA, 333 bp, Homo sapiens
  CDS (1)..(333)
atgaaatgcagctgggttatcttcttcctgatggcagtggttacaggggtcaattcaggctggaccctgaacagcgcgggctacctgctgggcccacatgccgttggcaaccacaggtcattcagcgacaagaatggcctcaccagcaagcgggagctgcggcccgaagatgacatgaaaccaggaagctttgacaggtccatacctgaaaacaatatcatgcgcacaatcattgagtttctgtctttcttgcatctcaaagaggccggtgccctcgaccgcctcctggatctccccgccgcagcctcctcagaagacatcgagcggtcctga

SEQ ID NO: 7 - protein, 110 aa, Homo sapiens
MKCSWVIFFLMAVVTGVNSGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 8 - protein, 64 aa, Homo sapiens
MTPPERLFLPRVRGTTLHLLLLGLLLVLLPGAQGGWTLNSAGYLLGPHAVGNHRSFSDKNGLTS

SEQ ID NO: 9 - protein, 68 aa, Homo sapiens
MGLRSWLAAPWGALPPRPPLLLLLLLLLLLQPPPPTWAGWTLNSAGYLLGPHAVGNHRSFSDKNGLTS

SEQ ID NO: 10 - protein, 49 aa, Homo sapiens
MKCSWVIFFLMAVVTGVNSGWTLNSAGYLLGPHAVGNHRSFSDKNGLTS

SEQ ID NO: 11 - protein, 123 aa, Homo sapiens
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 12 - protein, 107 aa, Homo sapiens
MKLWDVVAVCLVLLHTASAFPLPAGKRPPEAPAEDRSLGRRRAPFALSSDSNMPEDYPDQFDDVMDFIQATIKRLKRGWTLNSAGYLLGPHAVGNHRSFSDKNGLTS

SEQ ID NO: 13 - protein, 19 aa, Homo sapiens
MDCTWRILFLVAAATGTHA

SEQ ID NO: 14 - protein, 19 aa, Macaca mulatta
MKHLWFFLLLVAAPRWVLS

SEQ ID NO: 15 - protein, 19 aa, Callithrix jacchus
MDWTWRIFLLVATATGAHS

SEQ ID NO: 16 - protein, 19 aa, Mus musculus
MKCSWVIFFLMAVVTGVNS

SEQ ID NO: 17 - protein, 19 aa, Sus scrofa
MEFRLNWVVLFALLQGVQG

SEQ ID NO: 18 - protein, 19 aa, Rattus norvegicus
MKCSWIILFLMALTTGVNS

SEQ ID NO: 19 - protein, 26 aa, Homo sapiens
MATGSRTSLLLAFGLLCLSWLQEGSA

SEQ ID NO: 20 - protein, 18 aa, Rattus norvegicus
MKWVTFLLLLFISGSAFS

SEQ ID NO: 21 - protein, 19 aa, Rattus norvegicus
MKWVTFLLFLLFISGDAFA

SEQ ID NO: 22 - DNA, 227 bp, Salmo salar
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 23 - DNA, 228 bp, Salmo salar
ttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 24 - protein, 340 aa, Salmo salar
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 25 - protein, 340 aa, Salmo salar
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 26 - protein, 340 aa, Salmo salar
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCEKLVEGYPKRLTQVKQFKGNATKY

SEQ ID NO: 27 - protein, 123 aa, Sus scrofa
MPRGCALLLASLLLASALSATLGLGSPVKEKRGWTLNSAGYLLGPHAIDNHRSFHDKYGLAGKRELEPEDEARPGGFDRLQSEDKAIRTIMEFLAFLHLKEAGALGRLPGLPSAASSEDAGQS

SEQ ID NO: 28 - protein, 123 aa, Bos taurus
MPRGSVLLLASLLLAAALSATLGLGSPVKEKRGWTLNSAGYLLGPHALDSHRSFQDKHGLAGKRELEPEDEARPGSFDRPLAENNVVRTIIEFLTFLHLKDAGALERLPSLPTAESAEDAERS

SEQ ID NO: 29 - protein, 123 aa, Homo sapiens; identical to SEQ ID NO: 11

SEQ ID NO: 30 - protein, 124 aa, Rattus norvegicus
MARGSVILLAWLLLVATLSATLGLGMPTKEKRGWTLNSAGYLLGPHAIDNHRSFSDKHGLTGKRELPLEVEEGRLGSVAVPLPESNIVRTIMEFLSFLHLKEAGALDSLPGIPLATSSEDLEQS

SEQ ID NO: 31 - protein, 124 aa, Mus musculus
MARGSVILLGWLLLVVTLSATLGLGMPAKEKRGWTLNSAGYLLGPHAIDNHRSFSDKHGLTGKRELQLEVEERRPGSVDVPLPESNIVRTIMEFLSFLHLKEAGALDSLPGIPLATSSEDLEKS

SEQ ID NO: 32 - protein, 29 aa, Sus scrofa
GWTLNSAGYLLGPHAIDNHRSFHDKYGLA

SEQ ID NO: 33 - protein, 29 aa, Bos taurus
GWTLNSAGYLLGPHALDSHRSFQDKHGLA

SEQ ID NO: 34 - protein, 30 aa, Homo sapiens
GWTLNSAGYLLGPHAVGNHRSFSDKNGLTS

SEQ ID NO: 35 - protein, 29 aa, Rattus norvegicus
GWTLNSAGYLLGPHAIDNHRSFSDKHGLT

SEQ ID NO: 36 - protein, 29 aa, Mus musculus
GWTLNSAGYLLGPHAIDNHRSFSDKHGLT

SEQ ID NO: 37 - protein, 130 aa, Rattus norvegicus
MNLPSTKVPWAAVTLLLLLLLPPALLSLGVDAQPLPDCCRQKTCSCRLYELLHGAGNHAAGILTLGKRRPGPPGLQGRLQRLLQANGNHAAGILTMGRRAGAELEPYPCPGRRCPTATATALAPRGGSRV

SEQ ID NO: 38 - protein, 130 aa, Mus musculus
MNFPSTKVPWAAVTLLLLLLLPPALLSLGVDAQPLPDCCRQKTCSCRLYELLHGAGNHAAGILTLGKRRPGPPGLQGRLQRLLQANGNHAAGILTMGRRAGAELEPHPCSGRGCPTVTTTALAPRGGSGV

SEQ ID NO: 39 - protein, 131 aa, Homo sapiens
MNLPSTKVSWAAVTLLLLLLLLPPALLSSGAAAQPLPDCCRQKTCSCRLYELLHGAGNHAAGILTLGKRRSGPPGLQGRLQRLLQASGNHAAGILTMGRRAGAEPAPRPCLGRRCSAPAAASVAPGGQSGI

SEQ ID NO: 40 - protein, 131 aa, Sus scrofa
MNPPFAKVSWATVTLLLLLLLLPPAVLSPGAAAQPLPDCCRQKTCSCRLYELLHGAGNHAAGILTLGKRRPGPPGLQGRLQRLLQASGNHAAGILTMGRRAGAEPAPRLCPGRRCLAAAASSVAPGGRSGI

SEQ ID NO: 41 - protein, 33 aa, Rattus norvegicus
QPLPDCCRQKTCSCRLYELLHGAGNHAAGILTL

SEQ ID NO: 42 - protein, 33 aa, Mus musculus; identical to SEQ ID NO: 41

SEQ ID NO: 43 - protein, 33 aa, Homo sapiens; identical to SEQ ID NO: 41

SEQ ID NO: 44 - protein, 33 aa, Sus scrofa; identical to SEQ ID NO: 41

SEQ ID NO: 45 - protein, 28 aa, Rattus norvegicus
RPGPPGLQGRLQRLLQANGNHAAGILTM

SEQ ID NO: 46 - protein, 28 aa, Mus musculus
RPGPPGLQGRLQRLLQANGNHAAGILTM

SEQ ID NO: 47 - protein, 28 aa, Homo sapiens
RSGPPGLQGRLQRLLQASGNHAAGILTM

SEQ ID NO: 48 - protein, 28 aa, Sus scrofa
RPGPPGLQGRLQRLLQASGNHAAGILTM

SEQ ID NO: 49 - protein, 97 aa, Macaca mulatta
MLGSKRLGLSGLTLALSLLVCLGALAEAYPSKPDNPGEDAPAEDMARYYSALRHYINLITRQRYGKRSSPETLISDLLMRESTENVPRTRLEDPSMW

SEQ ID NO: 50 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 51 - protein, 98 aa, Rattus norvegicus
MMLGNKRMGLCGLTLALSLLVCLGILAEGYPSKPDNPGEDAPAEDMARYYSALRHYINLITRQRYGKRSSPETLISDLLMRESTENAPRTRLEDPSMW

SEQ ID NO: 52 - protein, 97 aa, Mus musculus
MLGNKRMGLCGLTLALSLLVCLGILAEGYPSKPDNPGEDAPAEDMARYYSALRHYINLITRQRYGKRSSPETLISDLLMKESTENAPRTRLEDPSMW

SEQ ID NO: 53 - protein, 36 aa, Macaca mulatta
YPSKPDNPGEDAPAEDMARYYSALRHYINLITRQRY

SEQ ID NO: 54 - protein, 36 aa, Homo sapiens; identical to SEQ ID NO: 53

SEQ ID NO: 55 - protein, 36 aa, Rattus norvegicus; identical to SEQ ID NO: 53

SEQ ID NO: 56 - protein, 36 aa, Mus musculus; identical to SEQ ID NO: 53

SEQ ID NO: 57 - DNA, 25 bp, Homo sapiens
tataggatccccgcagctcaagatg

SEQ ID NO: 58 - DNA, 30 bp, Homo sapiens
cgtttttttcttgacggcgaccagagcccc

SEQ ID NO: 59 - DNA, 31 bp, Homo sapiens
gtcgccgtcaagaaaaaaacgaggctggacc

SEQ ID NO: 60 - DNA, 26 bp, Homo sapiens
tatactcgagcaggaatggctgactc

SEQ ID NO: 61 - DNA, 31 bp, Homo sapiens
tataggatccggacgggactttaagatgaag

SEQ ID NO: 62 - DNA, 36 bp, Homo sapiens
ccttttttttcttgaacgggtggcttgaataaaatc

SEQ ID NO: 63 - DNA, 30 bp, Homo sapiens
cccgttcaagaaaaaaaaggggctggaccc

SEQ ID NO: 64 - DNA, 36 bp, Homo sapiens
tatactcgagtcagctggtgaggccattcttgtcgc

SEQ ID NO: 65 - DNA, 31 bp, Homo sapiens
ggtgaattcgggctggaccctgaacagcgcg

SEQ ID NO: 66 - DNA, 40 bp, Homo sapiens
tatactcgagcaggaatggctgactctgcataaattggcc

SEQ ID NO: 67 - DNA, 37 bp, Homo sapiens
tataggatccgccaccatgaaatgcagctgggttatc

SEQ ID NO: 68 - DNA, 31 bp, Homo sapiens
gggtccagcccgaattcacccctgtagaaag

SEQ ID NO: 69 - DNA, 30 bp, Homo sapiens
ggcccaggggggctggaccctgaacagcgc

SEQ ID NO: 70 - DNA, 76 bp, Homo sapiens
cgggatccatgacaccacctgaacgtctcttcctcccaagggtgcgtggcaccaccctacacctcctccttctggg

SEQ ID NO: 71 - DNA, 64 bp, Homo sapiens
gggtccagcccccctgggccccaggcagcagaaccagcagcagccccagaaggaggaggtgtag

SEQ ID NO: 72 - DNA, 30 bp, Homo sapiens
gacctgggcgggctggaccctgaacagcgc

SEQ ID NO: 73 - DNA, 84 bp, Homo sapiens
cgggatccatgggcctgaggagctggctcgccgccccatggggcgcgctgccgcctcggccaccgctgctgctgctcctgctgc

SEQ ID NO: 74 - DNA, 68 bp, Homo sapiens
gggtccagcccgcccaggtcggaggcggcggctgcagcaggagcagcagcagcaggagcagcagcagc